(19) United States
(12) Patent Application Publication
(10) Pub. No.: US 2026/0265285 A1
(43) Pub. Date: Sep. 10, 2026

(54) PRODUCTION OF A FUNGAL FERMENTATION MEDIUM FROM BREWER'S SPENT GRAIN

(71) Applicant: MUSHLABS GMBH, Hamburg (DE)

(72) Inventors: Thibault GODARD, Hamburg (DE); Daniel Alejandro DELGADO MONTUFAR, Hamburg (DE); Tripti SHARMA, Hamburg (DE); Wassim W. AYASS, Hamburg (DE)

(21) Appl. No.: 18/875,561

(22) PCT Filed: Jun. 29, 2023
(86) PCT No.: PCT/EP2023/067939

§ 371 Date: Dec. 16, 2024

(30) Foreign Application Priority Data
Jun. 29, 2022 (EP) 22182012.9

(51) Int. Cl. C07H 1/08; A01G 18/20; A23L 31/00; C07H 3/02; C12P 19/02; C12P 19/14

(52) U.S. Cl. CPC C07H 1/08; A01G 18/20; A23L 31/00; C07H 3/02; C12P 19/02; C12P 19/14; C12P 2203/00

(57) ABSTRACT

The present invention relates to a method for the production of a fungal fermentation medium from brewer's spent grain (BSG) and to a fermentation medium obtainable accordingly, to a method for production of a fungal biomass by submerged fermentation of at least one fungal strain and to a fungal biomass obtainable accordingly, and to a food product obtainable by using the instant fungal biomass of the invention.

### RELATED APPLICATIONS

This application is a 35 U.S.C. § 371 filing of International Patent Application No. PCT/EP2023/067939, filed Jun. 29, 2023, which claims priority to European Patent Application No. 22182012.9, filed Jun. 29, 2022, the entire disclosures of which are hereby incorporated herein by reference.

### FIELD OF THE INVENTION

The present invention relates to a method for the production of a fungal fermentation medium from brewer's spent grain (BSG) and to a fermentation medium obtainable accordingly, to a method for production of a fungal biomass by submerged fermentation of at least one fungal strain and to a fungal biomass obtainable accordingly, and to a food product obtainable by using the instant fungal biomass of the invention.

### BACKGROUND OF THE INVENTION

In recent years, production of food from animals has been receiving attention because of its unsustainability as well as rising concerns about animal welfare. In the context of climate change, many plant-based meat-alternatives have emerged with the aim to cut down CO₂ emissions and reduce animal suffering. However, these products are currently produced from three major monocrops (soy, pea and rice) whose culture requires a lot of land and water, heavily relies on chemical agents (pesticides and fertilizers) and generates a lot of wastes as only protein isolated from these crops is used for production of meat alternatives. In addition, these isolates have a strong bitter taste and no intrinsic texture and therefore their use in foods requires further processing steps as well as the addition of further ingredients, including but not limited to flavouring agents, texturizers, and/or colorants. Hence, plant-based alternatives are not necessarily healthy, and their production induces other environmental issues such as deforestation, significant reduction of biodiversity, soil pollution, and/or water contamination. It is to be noted that the present inventors are able to use the waste side streams as described hereinabove in the process as described herein and accordingly contribute to the improvement of the sustainability of these plant-based processes.

Production of food using fermentation processes seems to address several of these drawbacks. It enables a better use of land as fermenters can be scaled vertically and allows for the production of food locally in cities or villages. Moreover, they require less water per kilo product than plant protein, and with ongoing development and improvement of filtration and treatment technologies, this water could be recycled in the process. Herein disclosed is the production of fungal mycelium as new food product using a novel fermentation process wherein the growth medium as well as the final product are at least partially produced using lignocellulosic materials, e.g. industrial and/or agricultural sidestreams as raw material. In that sense, the process described herein contributes to the efforts to build a circular economy wherein industrial, food and agricultural wastes are reduced to a minimum and resources are used to their fullest extent. Another advantage of fungal fermentation over production of conventional plant isolates is comprised within the obtained raw material—fungal biomass—that per nature already has a desired fibrous texture and brings a balanced nutritional profile with complete proteins but also dietary fibres, vitamins and micronutrients that provide consumers with a healthy product. In particular, the use of mycelium isolated from the fruiting bodies from known edible mushrooms additionally brings a typical mushroom umami taste specific to this group, varying a bit between the species (e.g. morel, truffle or button mushroom) and enables the production of clean and tasty products with a very short list of ingredient.

The intensifying demand for a sustainable food supply chain and managing GHG emissions by looking for food alternatives are critical challenges across the globe. The use of renewable and waste lignocellulosic biomass as a raw material to produce food, creates a greener roadmap to achieve environmentally and economically sustainable processes.

Brewer's spent grain (BSG) is a lignocellulosic waste generated as a byproduct by the brewing industry. BSG is obtained as a solid residue after wort production in the brewing process. The product is initially wet, with a short shelf-life, but can be dried and processed in various ways. BSG, being a lignocellulosic material is rich in carbohydrates (hexoses, pentoses, and lignin) and hence, it is worth it to utilize the sugars locked within its structure. The use of BSG as a byproduct mitigates the issues associated with its disposal in the environment and aids in the circular economy.

As it is apparent to the skilled person, the carbohydrates available in the BSG are present in the form of fibres, cellulose and hemicellulose, and accordingly it has been recognized to be challenging to obtain said carbohydrates from the BSG.

DE10201410884 describes a process for deodorising lignin comprising the step of extracting a lignocellulosic substrate with a supercritical fluid or supercritical fluid mixture.

DE1020161 10653 relates to a food-product/fermentation product that comprises mycelia of fungi.

CN101838673A discloses the fermentation of a fungus of the Basidomycota family in a liquid fermentation media complemented with rice distiller grain.

CN1078872A discloses a method for the preparation of a drink comprising the cultivation of a fungus in a fermentation media comprising amongst other components vinasse.

WO 2017/208255A1 relates to a method of preparing edible fungi (of the phylum Ascomycota) by cultivation in media comprising vinasse.

WO2002090527A1 relates to a method of preparation of edible fungi (e.g. Fusarium species).

WO2017/181085A1 discloses certain methods for the production of fungal mycelia.

WO 2019/046480A1 relates to the preparation of edible filamentous fungal formulation by growing filamentous fungal biomats.

RU 2006/126554 relates to a method of producing food and feed biomass on nutrient media based on waste from distillery stillage production, which involves the sequential cultivation of baker's yeast Saccharomyces cerevisiae and edible basidiomycetes, for example, selected from the group including Pleurotus ostreatus, Pleurotus pulmonarius, among others.

U.S. Pat. No. 5,846,787 discloses a process for the treatment of cellulose containing material.

Papadaki (doi: 10.3390/microorganisms7070207) discloses the cultivation of Pleurotus species (P. pulmonarius and P. ostreatus) by solid state fermentation and semiliquid fermentation using grape pomace as sidestream.

Kim Min-Keun et al. (Korean Journal of Mycology, DOI: 10.4489/KJM.2012.40.1.049) discloses development of the optimal media for mycelial culture of Pleurotus eryngii using the hot water extract of raw materials. Described process is solid state fermentation for the production of fruiting bodies, wherein no steam is used.

Platt M. W. et al (Eur. J. Appl. Microbiol. Biotechnol vol. 17, pages 140-142, 1983) disclose increased degradation of straw by Pleurotus ostreatus sp. ‘florida’. Disclosed process is a solid state fermentation.

Beltran-Garcia M. J. et al. (Revista de la Sociedad Quimica de Mexico, vol 45, pages 77-81, 2001) disclose that lignin degradation products from corn stalks enhance notably the radial growth of basidiomycete mushroom mycelia, which is performed as solid state fermentation.

Document CN 108 203 693 A discloses certain Rhizopus oryzae seed culture medium.

Document KR 2013/0057507 discloses certain method of cultivation of Cordyceps militaris.

Document ES 2′370′215 discloses certain means of fungal cultivation.

Sidana Arushdeep et al. (Chinese Journal of Biology, vol 2014, pages 1 to 5) discloses sugarcane bagasse as a potential medium for fungal cultures.

Document U.S. Pat. No. 9,206,446 discloses certain extraction method from plant biomass.

Kemppainen et al. (Appl. Biochem. Biotechnology, 16 Apr. 2016, doi: 10.1007/s12010-016-2085-9) discloses certain aspects of steam explosion of brewer's spent grain.

Parchami et al. (Bioresource Technology 337 (2021) 125409—doi: 10.1016/j.biortech.2021.125409) discloses certain aspects of starch and protein recovery from brewer's spent grain using hydrothermal pretreatment and their conversion to edible filamentous fungi.

### SUMMARY OF THE INVENTION

Particularly desirable are means and methods that utilize lignocellulosic material, preferably agricultural and/or industrial waste, herein industrial and/or agricultural side stream(s), in particular brewer's spent grain, as they are cost effective and more sustainable. Further particularly desirable are methods that lead to obtaining the fungal biomass, and consequently the food product, with amino acid composition reflecting that of a complete protein according to FAO definition (www.fao.org, https://en.wikipedia.org/wiki/Complete_protein).

Further particularly desirable is a method for production of the fungal biomass that is resistant to contamination with other microorganisms, for example with bacteria. Accordingly, a fungal fermentation medium resistant to contamination with bacteria, obtainable by using lignocellulosic material, preferably an industrial and/or agricultural side stream(s) (i.e. waste products), in particular brewer's spent grain, is particularly desirable.

It was an object of the present invention to provide improved means and methods for the production of a fungal-based food product, methods for the production of a fungal fermentation medium from a lignocellulosic material, preferably from agricultural and/or industrial sidestream(s) as well as methods and means for the production of fungal biomass for the use in the production of fungal-based food products.

The present inventors have surprisingly found that brewer's spent grain can be extracted according to the methods disclosed herein yielding a fungal fermentation medium (upon supplementing the extract with at least one non-carbohydrate nutrient for fungal cultivation, as required) with high-sugar content, wherein the sugar content is mostly C5-complex sugars. Accordingly, the medium is fungal-specific as C5-complex sugars cannot support the growth of the most bacteria, and thus resistant to contamination. Noteworthy, the carbohydrates available in the BSG could be extracted according to the methods of the present invention, despite being present in the form of fibres, cellulose and hemicellulose, which makes the extraction process more difficult.

Accordingly, the problem described herein is solved by the embodiments described in the following and as characterized in the claims.

The invention is summarized in the following embodiments.

In a first embodiment, the present invention relates to a method for the production of a fungal fermentation medium from brewer's spent grain (BSG), the method comprising: (a1) extracting C5-sugars from the lignocellulosic material comprised in BSG via a steam pretreatment, followed by a washing step with liquid water at a temperature of not more than 50° C., and (b) combining the so obtained extract with at least one non-carbohydrate nutrient for fungal cultivation.

In a second embodiment, the present invention relates to method for the production of a fungal fermentation medium from brewer's spent grain (BSG), the method comprising: (a2) extracting C5 sugars from lignocellulosic material comprised in BSG via a liquid extraction treatment with water at temperature of between 145° C. and 155° C. and/or for a time up to 70 minutes, preferably for a time up to 50 minutes, preferably at the pressure of 30 to 50 bar, and (b) combining the so obtained extract with at least one non-carbohydrate nutrient for fungal cultivation.

In a third embodiment, the present invention relates to a fungal fermentation medium, obtainable according to the method for the production of a fungal fermentation medium from brewer's spent grain of the present invention.

In a fourth embodiment, the present invention relates to use of the medium of the present invention in the fungal culture.

In a fifth embodiment, the present invention relates to a method for producing a fungal biomass, the method comprising the step of submerged fermentation of at least one fungal strain using the fungal fermentation medium of the present invention.

In a sixth embodiment, the present invention relates to a fungal biomass produced according to the method for producing a fungal biomass of the present invention.

In a seventh embodiment, the present invention relates to use of fungal biomass of the present invention in production of a fungal-based food product.

In an eighth embodiment, the present invention relates to a fungal-based food product prepared using the fungal biomass of the present invention.

### BRIEF DESCRIPTION OF FIGURES

FIG. 1 shows the workflow of the method for production of a fungal fermentation medium from BSG, the method comprising step (a1) of steam pretreatment followed by washing with liquid water. Herein, the thermal hydrolysis followed by the extraction may also be referred to as steam pretreatment followed by washing with liquid water.

FIG. 2 shows the workflow of the method for production of a fungal fermentation medium from BSG, the method comprising step (a2) of hot liquid water treatment (indicated in the Figure as thermal hydrolysis/extraction).

FIG. 3 shows summary of the contamination experiments performed with the fungal fermentation media of the present invention.

FIG. 4 shows the concentrations of sugars extracted achieved under the same steam treatment conditions, but with different washing water temperatures.

### DETAILED DESCRIPTION OF THE INVENTION

The invention will be described in detail in the following. It is to be understood that, unless indicated to the contrary, all disclosed features can be combined with each other.

In one embodiment, the present invention relates to a method for the production of a fungal fermentation medium from brewer's spent grain (BSG).

Brewer's spent grain is preferably understood as a leftover or by-product of brewing industry. Preferably, spent grain is a material that remains after the mashing step and has a dry matter content of preferably between 10% and 30%. However, the dry matter content as recited herein is not meant to be limiting, as the skilled person is aware that dry matter content can be increased in preprocessing, for example by pressing, by drying or by other methods that are known to skilled person. Furthermore, the spent grain originating from other industries (for example spent grain obtainable as a byproduct of production of foodstuffs) can also be used within the scope of the present invention.

Preferably, brewer's spent grain that is suitable for use in the method of the present invention is characterized by a particle size distribution comprising a maximum between 0.3 to 1 mm, preferably a particle size referred to a coarse particle size by the skilled person, preferably with a maximum between 0.4 to 0.8 mm. Preferably, particle size of the brewers spent grain is determined in the sieving process that leads to production of said brewer's spent grain. Preferably, a particle size distribution comprises a maximum between 2.0 and 4.0 mm, and a second maximum between 1.0 and 2.0 mm without any pretreatment. Upon mechanical treatment, a particle size distribution comprises a single maximum at not more than 1.0 mm is used. Accordingly, as discussed in the literature, (Ozturk et al., J. Inst. Brew. 108(1):23-27, 2002), the brewer's spent grain may be upon grinding sifted through a series of sieves having apertures of 850, 425 and 212 μm. Depending on the fraction, the Brewer's spent grain preparations may be considered coarse (425-850 μm), medium (212-425 μm) and fine (<212 μm). As demonstrated in the Examples section, the skilled person will be in position to determine the particle size distribution also by using a different set of sieves than these disclosed hereinabove. It is preferred to use a set of sieves according to ASTM standards. It is further to be understood that the skilled person is in position to implement the method of the present invention also for other particle size distributions, for example for a particle size distribution comprising a maximum between 2.0 and 4.0 mm. The skilled person further recognizes that the spent grain, without any further mechanical (pre)treatment, has a particle size distribution comprising a maximum between 2.0 and 4.0 mm, and a second maximum between 1.0 and 2.0 mm. Upon mechanical treatment, it is common that an obtained particle size distribution will comprise a single maximum at not more than 1.0 mm.

Preferably, the BSG comprises between 20% and 25% w/w cellulose (preferably 22%), between 23 and 28% w/w hemicellulose (preferably 25.8%), and/or between 20% and 30% w/w protein (preferably 25% w/w protein). These numbers are understood to refer to the contents of the BSG with regard to its dry mass.

The methods of the present invention are disclosed and described herein in the context of brewer's spent grain. However, it is conceivable to the skilled person that said methods may also be applicable to any other lignocellulosic material. The lignocellulosic material is preferably herein defined as a material that comprises dry plant matter. Preferably, said lignocellulosic material comprises cellulose, hemicellulose and lignin. Preferably, at least one lignocellulosic material is at least one industrial and/or agricultural side stream, as defined herein. Further preferably, said lignocellulosic material is preferably solid.

Examples of the lignocellulosic material include spent grain, cereal brans, cotton, cotton seed husks, bagasse, cocoa shells, cocoa, cocoa pods, cotton and oil press cakes from sunflower, peanut, hazelnut, palm oil, olive, shells and husks from nuts, grass and leaves waste, wood chips, coffee grounds, coffee husks, coffee silverskin, rapeseed and byproducts from the soy industry like soybean pulp (“okara”).

Preferred lignocellulosic materials include spent grain, cereal brans (also referred to as wheat brans) and oil press cakes. In one particular embodiment, particularly preferred lignocellulosic material is cereal brans. It is particularly preferred that water used for prehydrolysis of cereal brans with steam comprises diluted base, for example not more than 1% w/w of said base. Particular suitable is NaOH at a concentration of up to 0.2% w/w, in particular at a concentration of 0.2% w/w. Alternatively, particular suitable is NaOH at a concentration of between to 0.2% w/w and 0.8% w/w, more preferably at a concentration between 0.6% w/w and 0.8% w/w, in particular at a concentration between 0.7% w/w and 0.8% w/w. Particularly suitable is NaOH at 0.7% w/w, more preferably particularly suitable is NaOH at 0.8% w/w. In one alternative embodiment, particular suitable is NaOH at 0.2% w/w.

Accordingly, it is conceivable to the skilled person that in the method of the present invention, the brewer's spent grain can be replaced with a side stream selected from spent beer grain, spent grain (from industries beyond brewery), cereal brans, bagasse, cotton and oil press cakes from sunflower, hazelnut, shells and husks from nuts, grass and leaves waste, wood chips, coffee grounds, coffee husks, coffee silverskin, rapeseed and byproducts from the soy industry like soybean pulp (“okara”), banana leaves, banana peels, chicory roots, cassava peels, citrus pulp, cocoa, cocoa bean shell, cocoa mucilage, cocoa pod husks, coconut fibers, coconut husk, coconut shell, coffee pulp, corn cob, corn stover, cotton, cottonseed meal, cotton seeds, hemp, spent hop, pea by-products, peanut hulls, peanut meal, peanut, potato peel raw, potato tuber, eucalyptus bark, Lantana weed, switch grass, rice bran, rice husk, rice straw, spent sugar beet, sugar beet pulp, sawdust, sugarcane bagasse wet, walnut shells, wheat bran, wheat distillers grains, wheat germ, wheat straw, lupin seeds, chickpea bran, chickpea pod husks, chickpea straw, olive waste, grape marc, pear pulp, sorghum bran, sorghum germ, sorghum stalk, sorghum straw, sunflower waste, and tea waste. Furthermore, it is also conceivable to the skilled person that in the methods of the present invention, the brewer's spent grain can be replaced with peels or waste or pulp or pomaces of the following sidestreams: oat, pine tree, dates, apple, apricot, spent barley, broccoli, cabbage, carrot, turnips, eggplant, kiwi, melon, alfaalfa, pineapple, pomegranate, plum, watermelon, zucchini, asparagus, beetroot, cauliflower, garlic, onion, pumpkin, squash and/or tomato.

In one embodiment, method for the production of a fungal fermentation medium from brewer's spent grain (BSG) comprises the steps (a1) extracting C5-sugars from the lignocellulosic material comprised in BSG via a steam pretreatment, followed by a washing step with liquid water at a temperature of not more than 50° C., and (b) combining the so obtained extract with at least one non-carbohydrate nutrient for fungal cultivation.

C5-sugars, as defined herein, preferably refer to a fraction wherein at least 80% w/w of entire sugar content constitute pentoses (saccharides including polysaccharides comprised of sugar subunits of five carbon atom. It is noted that C5-sugars, defined herein as a fraction comprising sugars, may contain other sugars, in particular C6 sugars (sugars having 6 carbon atoms, also referred to as hexoses), as monomers and/or comprised within polysaccharides and/or oligosaccharides. Accordingly, other sugars beyond pentoses may also be extracted in step (a1) or step (a2) of the methods of the present invention.

According to the present invention, the conditions of the extraction, in particular the temperature, the pressure, and the time of the extraction is set so that preferably at least 10%, at least 20%, at least 30%, at least 40%, at least 50%, at least 60%, at least 70% or at least 80% of saccharides contained in the lignocellulosic material in brewer's spent grain is recovered in the aqueous extraction. As understood by the skilled person, the time of the extraction may depend on further conditions, in particular on the applied reactor (in particular in the context of available stirring, as discussed herein), as well as on the particular type of material used in extraction.

In step (a1), preferably the steam pretreatment is performed at a severity factor of between 2.9 and 3.3.

Severity factor is also referred to as log₁₀(Ro), wherein Ro is herein defined as:

$$Ro = {t}_{R} {e}^{\frac{T - 100}{14.75}}$$

wherein t_{R} is retention time (expressed in minutes), also understood as the time of the process, and T is temperature expressed in ° C.

The steam pretreatment in step (a1) may be performed at a severity factor of between 2.9 and 3.0, at a severity factor of between 3.0 and 3.1, at a severity factor of between 3.1 and 3.2, and/or at severity factor of between 3.2 and 3.3. Preferably, the steam pretreatment in step (a1) may be performed at a severity factor of between 3.1 and 3.3, more preferably the steam pretreatment in step (a1) may be performed at a severity factor of between 3.1 and 3.2.

In one embodiment, the steam pretreatment in step (a1) may be performed at a severity factor between 2.0 and 4.0, more preferably at a severity factor of between 2.5 and 3.3, even more preferably at a severity factor of between 2.9 and 3.3.

In one embodiment, the steam pretreatment in step (a1) may be performed at a severity factor of between 2.6 and 3.0 or of between 3.1 and 3.3.

During the steam pretreatment in step (a1) the lignocellulosic material comprised in BSG may be contacted with steam at a temperature of between 130° C. and 180° C. It is preferred that the steam pretreatment in step (a1) is performed at a temperature of between 160° C. and 180° C., more preferably at a temperature of between 165° C. and 175° C. Accordingly, the steam pretreatment in step (a1) of the present invention may be performed at 130, 131, 132, 133, 134, 135, 136, 137, 138, 139 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 or 180° C. Preferably, during the steam pretreatment in step (a1) the lignocellulosic material comprised in BSG is contacted with steam for a time up of to 30 minutes, preferably for a time of up to 15 minutes. It is preferred that the steam pretreatment in step (a1) is performed for at least 5 minutes. Preferably, during the steam pretreatment in step (a1) the lignocellulosic material comprised in BSG is contacted with steam at a temperature of between 165° C. and 175° C. and/or for a time of up to 15 minutes, preferably for a time of up to 12.5 minutes, more preferably for a time of up to 10 minutes, even more preferably for a time of up to 7.5 minutes, even more preferably for a time of up to 5 minutes, even more preferably for a time of up to 2.5 minutes, even more preferably for a time of up to 1 minute. Within the invention, extraction of step (a1) may thus preferably be performed for about 7.5 minutes, about 5 minutes, about 2.5 minutes or about 1 minute. More preferably, during the steam pretreatment in step (a1) the lignocellulosic material comprised in BSG is contacted with steam at a temperature of between 165° C. and 175° C. and for a time of up to 15 minutes. More preferably, during the steam pretreatment in step (a1) the lignocellulosic material comprised in BSG is contacted with steam at a temperature of between 165° C. and 175° C. and fora time of up to 10 minutes. Even more preferably, during the steam pretreatment in step (a1) the lignocellulosic material comprised in BSG is contacted with steam at a temperature of between 165° C. and 175° C. and for a time of up to 7.5 minutes, even more preferably for a time of up to 5 minutes, even more preferably for a time of up to 2.5 minutes, even more preferably for a time of up to 1 minute. Thus, as described herein, extraction of step (a1) may thus preferably be performed for about 7.5 minutes, about 5 minutes, about 2.5 minutes or about 1 minute. Preferably, the temperature of between 165° C. and 175° C. relates to a temperature of about 170° C., more preferably the temperature of between 165° C. and 175° C. relates to a temperature of 170° C. Preferably, step (a1) is to be performed at a pressure not exceeding 10 bar.

In one embodiment of the present invention, the water used for prehydrolysis with steam may comprise diluted acid, for example not more than 1% w/w of said acid, preferably in the range from 0.8% w/w to 1% w/w, more preferably in the range from 0.8% w/w to 0.9% w/w. Particularly suitable are preparations of H₂SO₄ at 0.2% w/w, or 0.4% w/w, or 0.8% w/w or 0.9% w/w. However, also encompassed by the present invention are embodiments wherein water comprises up to 2% w/w of said acid, e.g. of H₂SO₄. Particularly suitable are preparations of H₂SO₄ ranging between 0.4% w/w and 1.6% w/w, preferably ranging between 0.6% w/w and 1.6% w/w, more preferably ranging between 0.7% w/w and 1.5% w/w. In one particular embodiment, the concentration of acid (preferably H₂SO₄) is between 0.4 and 1% w/w, more preferably between 0.4 and 0.9% w/w, still more preferably between 0.5 and 0.9% w/w. In one particular embodiment, the concentration of acid (preferably H₂SO₄) is between 1.1 and 1.6% w/w. This is in particular applicable to an embodiment wherein the lignocellulosic material is brewer's spent grain.

In one embodiment of the present invention, the water used for prehydrolysis with steam may comprise diluted base, for example not more than 1% w/w of said base. Particular suitable is NaOH at a concentration of between to 0.2% w/w and 0.8% w/w, more preferably at a concentration between 0.6% w/w and 0.8% w/w, in particular at a concentration between 0.7% w/w and 0.8% w/w. Particularly suitable is NaOH at 0.7% w/w, more preferably particularly suitable is NaOH at 0.8% w/w. In one alternative embodiment, particular suitable is NaOH at 0.2% w/w.

In an alternative embodiment of the present invention, the water used for prehydrolysis with steam is replaced with a phosphate buffer of a pH ranging between 4 and 6, more preferably of a pH ranging between 4.5 and 5.5.

In step (a1), the steam pretreatment is followed by a washing step with liquid water at a temperature of not more than 50° C. It is preferred that the washing step with liquid water is performed with water at a temperature of not more than 40° C., preferably not more than 30° C., more preferably not more than 25° C. Preferably the liquid water used for washing step is at room temperature, i.e. between 20° C. and 25° C., preferably at a temperature of about 22° C., more preferably at a temperature of 22° C. In an alternative preferred embodiment, the liquid water used for washing step ranges between 30° C. and 50° C. (in particular between 30° C. and 49° C., preferably between 30° C. and 48° C.), even more preferably between 4° and 50° C. (in particular between 40° C. and 49° C., preferably between 40° C. and 48° C.). Thus, the washing step may be performed with liquid water a temperature of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50° C. The time of washing is preferably at least 15 minutes, more preferably at least 20 minutes, more preferably at least 25 minutes, even more preferably at least 30 minutes. Preferably, the time or the duration of the washing step ranges between 15 and 30 minutes, even more preferably between 20 and 30 minutes. Accordingly, the duration of the washing step may be about 20 minutes, about 25 minutes or about 30 minutes. Depending on the desired outcome, times below than 15 minutes are also possible and also considered to be encompassed by the present invention.

Preferably, the washing step with liquid water at a temperature of not more than 50° C., as described hereinabove, may be performed simultaneously or sequentially with the ultrasound treatment.

Preferably, the washing step according to the present invention is performed with water at a pH of between 1 and 8, preferably between 1 and 5, more preferably at a pH between 1 and 4, even more preferably at a pH between 1 and 3, still more preferably at a pH between 1 and 2. Alternatively, the washing step can be performed with water at a pH of between 10 and 14, more preferably at a pH between 11 and 13.5, even more preferably at a pH between 12 and 13.5, still more preferably at a pH between 12.5 and 13.5. It is well known by a person skilled in the art how to calculate the needed corresponding amount of particular acid or base to be added needed to reach a particular concentration of H₃O⁺ or OH⁻ i.e. a particular pH value.

Preferably, in the method for the production of a fungal fermentation medium from brewer's spent grain (BSG) the BSG is characterized by a final moisture content of between 50 wt % and 75 wt %. Accordingly, said final moisture content may be achieved by dewatering BSG by pressing the material to a final moisture content between 50 and 75 wt %, using the methods known to the skilled person. This however is not meant to be construed as limiting as it is further apparent to the skilled person that other means and method for dewatering said BSG can also be applied here.

Accordingly, in a preferred embodiment, the method for the production of a fungal fermentation medium from brewer's spent grain (BSG) further comprises the step of dewatering BSG by pressing the material to a final moisture content between 50 and 75 wt %, wherein said dewatering step precedes step (a1). This step is conventional and accordingly known to the skilled person and can be performed for example by using the screw press (or any other suitable press) to press the side stream. Alternatively, the lignocellulosic material, preferably the lignocellulosic may be dried, for example by using a fluid bed dryer. A convection oven may also be used for this purpose. Preferably in the method of the present invention the so obtained water is discarded and not used further in the method of the present invention.

The method for the production of a fungal fermentation medium may further comprise further steps of preprocessing of the brewer's spent grain. Preferably, preprocessing of said brewer's spent grain comprises altering the particle size or other mechanical properties of the spent grain. For example, preprocessing of spent grain may comprise grinding of the spent grain. This step is conventional and known to the skilled person.

It is further apparent to the skilled person that residue of brewer's spent grain obtained after step (a1) can further undergo pretreatment, e.g. to increase its accessible surface area by e.g. breaking down particles, e.g. by grinding, crushing, pulverization etc. Such a step, which is herein disclosed as an optional step, is known to the skilled person. Said step of pretreatment may also be referred to as mechanical pretreatment. In particular, the step of pretreatment involving breaking down particles, e.g. by grinding, crushing, pulverization may also be referred to as mechanical treatment or mechanical pretreatment. While the step of pretreatment is herein described as following step (a1), it can also be implemented at other stages of the method, for example the brewer's spent grain can undergo the mechanical pretreatment step as discussed herein before it is subject to step (a1). Preferably, each of these mechanical pretreatment procedures can be done using a shredder or a disc refiner. It will be recognizable to the skilled person that such a shredder or a disc refiner is preferably located at the extractor outlet (extractor is herein understood as a reactor in which the extraction processes, as described herein, are to be performed, the extractor outlet is defined as an opening in the cavity used to remove the solid residue after the extraction), wherein said shredder or said disc refiner is configured to process the brewer's spent grain so that the BSG surface area is further increased (i.e., particle sizes further reduced) for a higher sugar extraction at the washing step.

In step (b) of the method for the production of a fungal fermentation medium of the present invention, the extract obtained in step (a1) is combined with at least one non-carbohydrate nutrient for fungal cultivation.

Accordingly, the aqueous extract(s) of step (a1) obtained according to the present invention can be further supplemented with nitrogen source(s), carbon source(s), trace element(s), vitamin(s) and/or protein composition(s). The nitrogen sources as defined herein are preferably selected from ammonia, urea, yeast extract, malt extract, corn steep liquor and peptone. More preferably, the nitrogen source(s) are ammonia and/or urea. The carbon source(s) are preferably selected from glucose, fructose, sucrose, lactose, maltose, xylose, galactose, dextrose, glycerol, and molasses, more preferably the carbon source is glucose. Preferably, no carbon sources beyond those originating from BSG are added to the medium of the present invention. The trace element(s) as defined herein may include for example iron(III) salts, copper(II) salts, zinc salts, manganese(II) salts, molybdenum salts and/or cobalt(II) salts. Vitamins as defined herein preferably include vitamins that are beneficial for the growth of fungi on the medium obtainable according to the method of the present invention, for example folic acid, riboflavin, pantothenic acid or biotin. Protein composition may be further used to supplement the aqueous extract of (a1) of the present invention. Preferably, protein composition obtainable from proteins separated from the aqueous extract(s) of (a1) of the method of the present invention, preferably by flocculation or by precipitation with CO₂, are preferably used in the method of the present invention.

Preferably, according to step (b) the extract of step (a1) is preferably supplemented with at least one nitrogen source, as described hereinabove.

It is further preferred that in step (b) the extract of step (a1) is substantially undiluted. Herein, the term substantially undiluted preferably means not more than 15% dilution (which is preferably to be understood that the final concentration of the extract of point (a1) in the medium of step (b) is not decreased by more than 15% upon step (b), more preferably not more than 10% dilution. Accordingly, preferably said extract of step (a1) constitutes at least 85% v/v of the entire medium obtained in step (b), preferably said extract of step (a1) constitutes at least 90% of the entire medium obtained in step (b), more preferably said extract of step (a1) constitutes 90 to 99% v/v of the entire medium obtained in step (b), even more preferably wherein said extract of step (a1) constitutes 94 to 98% v/v of the entire medium obtained in step (b).

Preferably, in the method of producing a fungal fermentation medium of the present invention, step (b) comprises addition of solid substances, preferably addition of solid nitrogen source(s), as known to the skilled person.

The present inventors have found that, preferably, the medium obtainable in the method of the present invention wherein method includes step (a1), i.e. the method involves steam pretreatment, is substantially free of fungal growth inhibitors, e.g. hydroxymethylfurfural or furfural. Accordingly, the medium obtainable as described herein is preferably capable of supporting fungal growth without further dilution steps to reduce the concentration of fungal growth inhibitors.

It is recognized to the skilled person that if the medium obtainable according to the present invention is subjected to postprocessing by removal of the fungal growth inhibitors, the use of dilution step which is meant to reduce their concentration to acceptable level may be avoided.

Further examples of fungal growth inhibitors that may be formed in the extraction processes are weak acids (e.g. acetic acid, formic acid, levulic acid), furans (furfural, hydroxymethylfurfural, 2-furoic acid) and/or phenolics (vanillin, syringaldehyde, ferulic acid, coumaric acid, coniferylalcohol, eugenol, acetovanillin, feruloylamide, coumaryl amide).

Undesired presence of furfural and/or hydroxymethylfurfural and/or other undesired compounds may be avoided, as described above. Furthermore, undesired furfural and/or hydroxymethylfurfural and/or other undesired compounds may be removed, for example by gas stripping, by heteroazeotropic distillation or by liquid-liquid extraction. For example, in one embodiment, furfural and/or other fungal growth inhibitors may be removed by using a suitable membrane. Optionally, furfural may be recovered and used for other industrial applications. It is preferred that upon extraction as described herein the concentration of furfural does not exceed 0.6 g/1, preferably the concentration of furfural does not exceed 0.2 g/L, preferably the concentration of furfural does not exceed 0.15 g/L, more preferably the concentration of furfural does not exceed 0.1 g/L, even more preferably the concentration of furfural does not exceed 0.05 g/L, still more preferably the concentration of furfural does not exceed 0.01 g/L. Preferably, when extraction is performed according to the methods of the present invention, the concentration of furfural in the final extract is as described herein.

Accordingly, the present invention further relates to an embodiment wherein in the obtained medium before step (b) the concentration of furfural does not exceed 0.6 g/L, preferably the concentration of furfural does not exceed 0.2 g/L, preferably the concentration of furfural does not exceed 0.15 g/L, more preferably the concentration of furfural does not exceed 0.1 g/L, even more preferably the concentration of furfural does not exceed 0.05 g/L, still more preferably the concentration of furfural does not exceed 0.01 g/L.

In one embodiment of the method for the production of a fungal fermentation medium from brewer's spent grain (BSG) of the invention, the method comprises a step (a2) which comprises liquid extraction treatment of BSG with water.

Accordingly, in step (a2) of the method of the invention C5 sugars from lignocellulosic material comprised in BSG are extracted via liquid extraction treatment with water.

Within the methods of the present invention, said extraction in step (a2) to obtain C5 may be performed at a temperature between 140° C. and 180° C. It is preferred that extraction in step (a2) is performed at a temperature between 145° C. and 175° C., more preferably at a temperature between 145° C. and 170° C., even more preferably at a temperature between 145° C. and 160° C., even more preferably at a temperature between 145° C. and 155° C. In one preferred embodiment of the invention, extraction may be performed at a temperature of about 150° C. Accordingly, within the present invention, extraction in step (a2) may be performed at 140, 141, 142, 143, 144, 145, 146, 147, 148, 149, 150, 151, 152, 153, 154, 155, 156, 157, 158, 159, 160,161, 162, 163, 164, 165, 166, 167, 168, 169, 170, 171, 172, 173, 174, 175, 176, 177, 178, 179 or 180° C.

As the skilled person is aware, temperature and pressure are linearly related. Within the present invention, the extraction of step (a2) to obtain C5 sugars may be performed at a pressure between 30 to 50 bar. Accordingly, the extraction of step (a2) may be performed at 30, 31, 32, 33, 34, 35, 36, 37, 38, 39, 40, 41, 42, 43, 44, 45, 46, 47, 48, 49 or 50 bar. It is preferred that the extraction of step (a2) is performed at 50 bar.

Within the methods of the invention including step (a2), the extraction is performed to obtain C5 sugars from lignocellulosic material comprised in BSG. Accordingly, extraction is performed for a minimal time that is sufficient to obtain C5 sugars. Within the present invention, the extraction is performed for up to 70 minutes. It is preferred that the extraction of step (a2) is performed for up to 50 minutes, more preferably for up to 30 minutes, more preferably for up to 25 minutes, more preferably for up to 15 minutes, more preferably for up to 10 minutes. It is most preferred that the extraction of step (a2) is performed for up to 5 minutes to obtain C5 sugars. Within the invention, extraction of step (a2) may thus preferably be performed for a time of 5 to 15 minutes, more preferably for about 15, about 10 or about 5 minutes. It is preferred that the extraction of step (a2) is performed for at least 5 minutes. Furthermore, said extraction may be performed at the pressure of 5 to 50 bar. It is preferred that said extraction is performed at a pressure of 10 to 50 bar, more preferably at a pressure of 30 to 50 bar. Preferably, the pressure and temperature are so selected that in the conditions of the extraction in step (a2) the water remains liquid.

Preferably, step (a2) of the method of the present invention is performed at a severity factor of between 2.9 and 3.3. Accordingly, step (a2) may be performed at a severity factor of between 2.9 and 3.0, at a severity factor of between 3.0 and 3.1, at a severity factor of between 3.1 and 3.2, and/or at severity factor of between 3.2 and 3.3. Preferably, the steam pretreatment in step (a2) may be performed at a severity factor of between 3.1 and 3.3, more preferably the steam pretreatment in step (a2) may be performed at a severity factor of between 3.1 and 3.2.

In one embodiment, step (a2) of the method of the present invention may be performed at a severity factor between 2.0 and 4.0, more preferably at a severity factor of between 2.5 and 3.3, even more preferably at a severity factor of between 2.9 and 3.3. Particularly preferred is an embodiment wherein step (a2) is performed at a severity factor of between 2.9 and 3.3 and at a temperature of between 145° C. and 175° C., preferably at a temperature of between 145° C. and 170° C., more preferably at a temperature of between 145° C. and 160° C., even more preferably at a temperature of 145° C. and 155° C., even more preferably at a temperature of about 150° C. Accordingly, in this particularly preferred embodiment, step (a2) of the present invention may be performed at a temperature of 145, 146, 147, 148, 149, 150, 151, 152, 153, 154, or 155° C. In an alternative embodiment, step (a2) of the method of the present invention may be performed between 130° C. and 140° C. In this particular embodiment, the pH of water used in step (a2) is preferably between 1.0 and 3.0.

Alternatively, it is preferred that the step (a2) of the method of the present invention is performed at a severity factor between 2.0 and 3.0.

In one embodiment of the present invention, the water used for liquid extraction treatment with water may comprise diluted acid, for example not more than 1% w/w of said acid preferably in the range from 0.8% w/w to 1% w/w, more preferably in the range from 0.8% w/w to 0.9% w/w. Particularly suitable are preparations of H₂SO₄ at 0.2% w/w, or 0.4% w/w, or 0.8% w/w or 0.9% w/w. However, also encompassed by the present invention are embodiments wherein water comprises up to 2% w/w of said acid, e.g. of H₂SO₄. This is in particular applicable to an embodiment wherein the lignocellulosic material is brewer's spent grain. Particularly suitable are preparations of acid, preferably H₂SO₄ ranging between 0.4% w/w, to 1.6% w/w, preferably ranging between 0.6% w/w to 1.6% w/w, more preferably ranging between 0.7% w/w and 1.5% w/w. In one particular embodiment, the concentration of acid (preferably H₂SO₄) is between 0.4 and 1% w/w, more preferably between 0.4 and 0.9% w/w, still more preferably between 0.5 and 0.9% w/w. In one particular embodiment, the concentration of acid (preferably H₂SO₄) is between 1.1 and 1.6% w/w. This is in particular applicable to an embodiment wherein the lignocellulosic material is brewer's spent grain. It is to be understood that this embodiment can be combined with the embodiment of the three preceding paragraphs, reciting preferred temperatures and severity factors for the (a2) step.

In one embodiment of the present invention, the water used for liquid extraction treatment with water may comprise diluted base, for example not more than 1% w/w of said base. Particular suitable is NaOH at a concentration of between to 0.2% w/w and 0.8% w/w, more preferably at a concentration between 0.6% w/w and 0.8% w/w, in particular at a concentration between 0.7% w/w and 0.8% w/w. Particularly suitable is NaOH at 0.7% w/w, more preferably particularly suitable is NaOH at 0.8% w/w. Alternatively, particularly suitable is NaOH at 0.2% w/w.

In an alternative embodiment of the present invention, the water used for liquid extraction treatment is replaced with a phosphate buffer of a pH ranging between 4 and 6, more preferably a pH ranging between 4.5 and 5.5.

Preferably, in the method of the present invention, the lignocellulosic material comprised in BSG to be extracted in step (a2) of said method is untreated, preferably wherein said material has not been dewatered.

Preferably, step (a2) of aqueous extraction of a lignocellulosic material, preferably industrial and/or agricultural side stream according to the present invention is performed with water at a pH of between 2.0 and 12.0, preferably 3.0 and 10.0, more preferably 4.0 and 8.0, even more preferably 5.0 and 8.0. However, it is also preferred that step (a2) of aqueous extraction of a lignocellulosic material, preferably industrial and/or agricultural side stream according to the present invention is performed with water at a pH of between 1.0 and 12.0, preferably 1.0 and 10.0, more preferably 1.0 and 8.0, even more preferably 1.0 and 8.0. The pH values as understood herein are measured under a pressure of 1.0 bar and temperature of 25° C., even though the extraction itself is performed under different conditions, as disclosed herein. Preferably, the pH is adjusted before the water is placed in contact with the at least one lignocellulosic material, preferably industrial and/or agricultural side stream. It is further understood herein, that addition of acid or base to water as described herein to a final concentration of more than 1% w/w is preferably to be avoided.

More preferably, step (a2) of aqueous extraction of a lignocellulosic material requiring an acid treatment, preferably industrial and/or agricultural side stream according to the present invention is performed with water at a pH of between 1 and 5, more preferably at a pH between 1 and 4, even more preferably at a pH between 1 and 3, still more preferably at a pH between 1 and 2. This is in particular applicable to an embodiment wherein the lignocellulosic material is brewer's spent grain. Alternatively, in the case of using base treatment, it is preferred that in step (a2) of aqueous extraction of a lignocellulosic material requiring a base treatment, preferably industrial and/or agricultural side stream according to the present invention, is performed with water at a pH of between 10 and 14, more preferably at a pH between 11 and 13.5, even more preferably at a pH between 12 and 13.5, still more preferably at a pH between 12.5 and 13.5. This is in particular applicable to an embodiment wherein the lignocellulosic material is wheat bran. It is well known by a person skilled in the art how to calculate the needed corresponding amount of particular acid or base to be added needed to reach a particular concentration of H₃O⁺ or OH⁻ i.e. a particular pH value.

Aqueous extraction step (a2) may be performed by any technical method, as known to the skilled person. For example, the aqueous extraction step (or each of the step(s)) can be performed as a batch process. Preferably however, aqueous extraction step(s) (a2) of the method of the present invention are to be performed as a continuous extraction process, as known to the skilled person.

Preferably, the batch extraction is performed in a closed system, wherein the spent grain and water are loaded just once at the beginning. The reactors are preferably pressurized and remaining closed until the mixture is cooled to at least 40° C. Continuous extraction is preferably performed in a fashion that the continuous feeding and retrieving of the feedstock is at the same speed rate, leading only to a pressure drop when materials are retrieved created in a steam explosion. A semi-continuous process, the feed and the reaction mixture enter and exit the system continuously. The feeding and retrieving of the feedstock are not at the same speed rate, the feedstock is fed in cycles.

As it is to be understood herein, the step of washing with water, as described in the step (a1), can also be performed as a batch process or as a semicontinuous process. Thus, in one embodiment, the step of washing with water in step (a1), as described herein, is performed as a batch process, as described herein. Preferably in the batch process, at least 50% C5-sugars is recovered, more preferably at least 60% C5-sugars is recovered. In one embodiment wherein the step of washing with water in step (a1) is performed as a batch process, as described herein, but in the presence of dilute acid (up to 2% w/w), at least 70%, preferably at least 80%, more preferably at least 90% C5-sugars is recovered. In one embodiment, the step of washing with water in step (a1), as described herein, is performed as a semicontinuous process, as described herein. Preferably, in a semicontinuous process, at least 80% C5-sugars are recovered, more preferably at least 90% C5-sugars are recovered, even more preferably at least 96% C5-sugars are recovered.

The method of the present invention for the production of a fungal fermentation medium from brewer's spent grain (BSG) comprising the step (a2) further comprises step (b), i.e. combining the so obtained extract with at least one non-carbohydrate nutrient for fungal cultivation. Step (b) in the herein described embodiment of the method is as described hereinabove, i.e. as for the method of the invention for the production of a fungal fermentation medium from brewer's spent grain comprising the step (a1).

Preferably, in the methods of the present invention substantially no sugar beyond that present in the extract of step (a1) or the extract of step (a2), as applicable is added to the medium. Substantially is herein preferably meant as not more sugar is added than an amount corresponding to 5% w/w of the sugar present in the extract of step (a1) or the extract of step (a2), more preferably not more sugar is added than an amount corresponding to 1% w/w of the sugar present in the extract of step (a1) or the extract of step (a2), even more preferably no additional sugar is added to the sugar present in the extract of step (a1) or the extract of step (a2), as applicable. Accordingly and preferably, in the methods of the present invention no sugar beyond that present in the extract of step (a1) or the extract of step (a2), as applicable is added to the medium.

Optionally, in the method of the present invention comprising the step (a1) or in the method of the present invention comprising the step (a2), the lignocellulosic material, preferably the brewer's spent grain, before being subjected to the methods for production of a fungal fermentation medium of the present invention, may undergo further pretreatment steps. Accordingly, the lignocellulosic material (e.g. comprised in brewer's spent grain, as described herein) as used herein can undergo pretreatment according to the method selected from washing, solvent-extraction, solvent-swelling, comminution, milling, dilute acid pretreatment, hot water pretreatment, alkaline pretreatment, lime pretreatment, wet oxidation, wet explosion, ammonia fiber explosion, organosolvent pretreatment, biological pretreatment, ammonia percolation, ultrasound, electroporation, microwave, supercritical CO₂, supercritical H₂O, ozone, and gamma irradiation. Preferably, no further preprocessing steps beyond described in step (a) are comprised in the method of the present invention for the production of a fungal fermentation medium.

As understood herein, in the method of the present invention comprising the step (a1) or in the method of the present invention comprising the step (a2), the extraction step (a1) or (a2), as applicable, of extraction of the lignocellulosic material comprised in brewer's spent grain, is performed in a suitable reactor, known to the skilled person. The at least one lignocellulosic material, preferably industrial and/or agricultural side stream is loaded into the said reactor at the solid load preferably between 5 and 70% weight/volume (w/w), preferably between 10 and 55% (w/w) and treated with water or with steam. The solid load as understood herein is defined as the ratio of weight of dry solid side stream (the material loaded) to the complete reaction volume (including the water used for extraction and the at least one solid side stream, and preferably expressed as percentage. The weight of material loaded is herein understood as the dry weight. The solid load as required in the present invention depends on the material loaded and the reactor characteristics. As understood to the skilled person, the amount of material loaded preferably should not affect the stirring and the heat transfer inside the reactor. It also depends on the amount of liquid extract that has to be recovered as well as its composition. As understood herein, the reactor can preferably be loaded up to 55% w/w with dry lignocellulosic biomass (also referred to as dry solid side stream). It is noted that in certain embodiments, alternatively, the reactor can be preferably loaded with wet lignocellulosic biomass. As known to the skilled person, the preferred load depends on the material, i.e. lignocellulosic material, preferably industrial and/or agricultural side stream.

Optionally, in the method of the present invention comprising the step (a1) or in the method of the present invention comprising the step (a2) the aqueous extract obtained in step (a1) or in step (a2) may undergo further processing. The processing the aqueous extract(s) obtained in (a1)/(a2) before the step (b), may preferably include separation of proteins from the aqueous extracts of (a1)/(a2). Any separation method suitable for the purpose and known to the skilled person can be used in the method of the present invention. Preferably, the said proteins are separated from the aqueous extract(s) preferably by flocculation or by precipitation with CO₂, preferably followed by mechanical separation, for example with a decanter centrifuge. Optionally, the so obtained proteins may be further prepared as a solution comprising not less than 50% w/w of polypeptides and/or amino acids. Further optionally, the so obtained proteins are hydrolyzed, preferably by using proteolytic enzymes, in particular selected from alcalases, papain, proteinase K, and trypsin. The proteolytic enzymes can be used, at a concentration of between 0.01% and 5% (w/w) (which is understood herein as a total concentration of all the enzymes used herein) and/or at a temperature of between 15 and 100° C. and/or for a time of between 0.5 and 96 hours. The so obtained solution(s) containing hydrolyzed protein(s) (i.e. polypeptide(s) and amino acids) can optionally be further used in step (b) of the method of the present invention. It is further noted that further processing of the aqueous extract obtained in step (a1) or in step (a2) any contaminants and/or fungal growth inhibitors may be removed.

The term “polypeptide” as used herein covers proteins, peptides and polypeptides, wherein said proteins, peptides or polypeptides may or may not have been post-translationally modified. Post-translational modification may for example be phosphorylation, methylation, and/or glycosylation. However, other post-translational modifications recognizable to the skilled person are also encompassed.

Preferably, in the method for producing the fungal fermentation medium according to the present invention, the proteins are not separated from the extract, i.e. from the product of the said method. The present inventors have found it to be beneficial to keep the proteins, as defined herein, in the medium as nitrogen source. As understood herein, the proteins may comprise amino acid, peptides and/or proteins.

Preferably, step (a1) or step (a2) in the methods for the production of a fungal fermentation medium is characterized by the solid load of a reactor of between 1 and 25%, preferably 1 and 15%, more preferably 4 and 14% w/w, even more preferably between 8 and 14% w/w, even more preferably between 10 and 14% w/w, even more preferably between 12 and 14% w/w. In one particular embodiment, the solid load of the reactor is of between 5 and 15%.

As understood herein, the product of the step (b) of the method of the present invention may be further processed. Optionally, the water contained in the product of step (b) may be removed, for example by spray-drying, drum-drying, belt-drying, or freeze-drying, yielding dried fungal fermentation medium, which as known to the skilled person may have improved shelf time. Optionally, the fungal fermentation medium of the present invention may be further sterilized or pasteurized within the scope of the method of the present invention. Optionally, the fungal fermentation medium obtainable according to the method of the present invention may be further supplemented, for example by salts (preferably sodium chloride, sodium nitrate, magnesium sulfate, calcium chloride, calcium carbonate, ammonium chloride, diammonium phosphate, ammonium sulfate, potassium phosphate, disodium phosphate, and/or monosodium phosphate), antibiotics, or by water. The water, as used herein, is preferably used to optimize the concentration of the components in the medium.

Further preferably, the pH of the fungal fermentation medium obtainable in the method for the production of a fungal fermentation medium from at least one lignocellulosic material, preferably industrial and/or agricultural side stream of the present invention can be set to a desired value preferably by addition of buffering agents. Particularly useful herein are citrate or phosphate buffer systems. Further, in the fermenter the pH can be adjusted by addition of the appropriate amount of urea, NaOH, ammonia, sulfuric acid, phosphoric acid, or hydrochloric acid.

Preferably, the fungal fermentation medium of the present invention comprises complex C5-sugars (also referred to as complex C5 polysaccharides). As understood herein, complex C5-sugars are C5-sugars that preferably in at least 60%, at least 70%, at least 80% or at least 90% are comprised of molecules comprising two or more sugar units.

Accordingly and preferably, complex C5-polysaccharides constitute at least 50% of all sugars in said medium, preferably wherein complex C5-polysaccharides constitute at least 65% of all sugars in said medium, more preferably wherein complex C5-polysaccharides constitute at least 80% of all sugars in said medium said medium. It is to be understood that the medium obtainable in the method of the present invention wherein the method comprises step (a′), will not necessarily fulfil this condition, as the product of step (a′) comprises C6 sugars. Accordingly and preferably, complex C5-polysaccharides constitute at least 50% of all sugars in the extract produced in step (a1) or in step (a2), preferably wherein complex C5-polysaccharides constitute at least 65% of all sugars in said extract, more preferably wherein complex C5-polysaccharides constitute at least 80% of all sugars in said extract.

Preferably, the medium obtainable according to the methods of the present invention is suitable for cultivation of at least one fungal strain selected from Pezizales, Boletales, Cantharellales, Agaricales, Polyporales, Russulales, Auriculariales, Sordoriales and Hypocreales, preferably at least one fungal strain selected from P. pulmonarius P. ostreatus, P. citrinopileatus, P. salmoneostramineus, M. rufobrunnea, M. esculenta, M. angusticeps and M. deliciosa, preferably selected from P. pulmonarius and M. rufobrunnea.

Also encompassed by the present invention are embodiments wherein the steam treatment followed by washing with liquid water in step (a1) is followed by liquid water treatment as in step (a2) of the obtained solid residue, only then followed by the step (b). Alternatively, step (a2) may be followed by step (a1) performed on the solid residue obtained after said step (a2), then followed by step (b). Further encompassed by the present invention are the methods which comprise step (a1), as defined herein, and/or step (a2), as defined herein, followed by step (b), as defined herein. According to the present inventors performing more than one step on BSG (or respectively a residue obtained in the precedent step) selected from step (a1) and step (a2), as described herein, allows recovering more sugar and avoids extensive production of fungal growth inhibitors.

In one embodiment, step (a1) of the steam treatment followed by washing with liquid water is followed by another step (a1) of the steam treatment followed by washing with liquid water, performed on the solid residue obtained in the first step (a1), only then followed by the step (b). Optionally, step (a′) may be performed before step (b).

In one embodiment, step (a2) of liquid water treatment is followed by another step (a2) of liquid water treatment, performed on the solid residue obtained in the first step (a2), only then followed by the step (b). Optionally, step (a′) may be performed before step (b).

In one embodiment, step (a1) of the steam treatment followed by washing with liquid water treatment is followed by another step (a2) of liquid water treatment, performed on the solid residue obtained in the first step (a1), only then followed by the step (b). Optionally, step (a′) may be performed before step (b).

In one embodiment, step (a2) of liquid water is followed by another step (a1) of the steam treatment followed by washing with liquid water, performed on the solid residue obtained in the first step (a2), only then followed by the step (b). Optionally, step (a′) may be performed before step (b).

In the method of the present invention, in step (a1) or step (a2) a solid lignocellulosic residue is generated. Said residue may be further used in the method of the present invention. Accordingly and preferably, the method for the production of a fungal fermentation medium from brewer's spent grain may further comprise the step (a′) of enzymatic hydrolysis of a solid lignocellulosic residue obtained in step (a1) or in step (a2) with cellulase, and separating a liquid product of hydrolysis from a solid residue. Accordingly, the lignocellulosic residue is loaded into the second reactor (preferably at a load of between 5 and 50% w/w) where it is hydrolysed with cellulase. Preferably, the step (a′) is performed at a temperature of between 15 and 100° C., more preferably at a temperature of between 4° and 80° C. Preferably, the step (a′) is performed at a pH between 3.0 and 8.0, more preferably at a pH between 4.0 and 7.0. Preferably, the step (a′) is performed for a time of between 10 and 200 hours.

Preferably, the solid lignocellulosic residue generated in step (a1) or step (a2) undergoes no further processing before it is subjected to step (a′).

Preferably, the extract obtained in step (a′) comprises C6 sugars. C6 sugars are preferably defined as hexoses (i.e. sugars comprising sugar units of six carbon atoms) or oligo and polysaccharides built of hexose units.

Preferably, the extract obtained in step (a′) is to be mixed with the extract obtained in step (a1) or with the extract obtained in step (a2). Preferably, the ratio of mixed extract in step (a1) to the extract of step (a2) is not meant to be particularly limited. Said ratio may depend on the requirements of particular medium production and will be a consequence of the solid load of the reactor applied for each of these steps.

In a further embodiment, the present invention relates to a fungal fermentation medium obtainable in the method for the production of a fungal fermentation medium of the present invention. The fungal fermentation medium of the present invention obtainable in the method for the production of a fungal fermentation medium from at least one lignocellulosic material, preferably industrial and/or agricultural side stream of the present invention may optionally further comprise nitrogen source(s), carbon source(s), trace element(s), vitamin(s) and/or protein composition(s). The nitrogen sources as defined herein are preferably selected from ammonia, urea, yeast extract, malt extract, corn steep liquor and peptone. More preferably, the nitrogen source(s) are ammonia and/or urea. The carbon source(s) are preferably selected from glucose, fructose, sucrose, lactose, maltose, xylose, galactose, dextrose, glycerol, and molasses, more preferably the carbon source is glucose or xylose. It is however preferred that no carbon sources beyond those obtained through the extraction of BSG are added herein. Accordingly, the invention encompasses preferably no addition of further sugars beyond those obtained in any one of steps (a1), (a2) and/or (a′) of the method of the present invention. The trace element(s) as defined herein may include for example iron(III) salts, copper(II) salts, zinc salts, manganese(II) salts, molybdenum salts and/or cobalt(II) salts. Vitamins as defined herein preferably include vitamins that are beneficial for the growth of fungi on the medium obtainable according to the method of the present invention, as defined herein.

Preferably, in the fungal fermentation medium of the present invention, substantially all the sugar originates from BSG. Substantially is herein preferably meant as not more sugar is added than an amount corresponding to 5% w/w of the sugar present in the extract of step (a1) and/or the extract of step (a2) and/or the extract of step (a′), more preferably not more sugar is added than an amount corresponding to 1% w/w of the sugar present in the extract of step (a1) and/or the extract of step (a2) and/or the extract of step (a′), even more preferably no additional sugar is added to the sugar present in the extract of step (a1) and/or the extract of step (a2) and/or the extract of step (a′), as applicable.

In the fungal fermentation medium of the present invention preferably complex C5-polysaccharides constitute at least 50% of all sugars in said medium, preferably complex C5-polysaccharides constitute at least 65% of all sugars in said medium, more preferably complex C5-polysaccharides constitute at least 80% of all sugars in said medium said medium. It is to be understood that the medium obtainable in the method of the present invention wherein the method comprises step (a′), will not necessarily fulfil this condition, as the product of step (a′) comprises C6 sugars. Accordingly and preferably, complex C5-polysaccharides constitute at least 50% of all sugars in the extract produced in step (a1) or in step (a2), preferably wherein complex C5-polysaccharides constitute at least 65% of all sugars in said extract, more preferably wherein complex C5-polysaccharides constitute at least 80% of all sugars in said extract.

In an embodiment of the present invention wherein the extract of step (a1) or the extract of step (a2) are mixed with the extract of step (a′), complex polysaccharides may constitute less than 50% of all sugars in the medium.

The fungal fermentation medium of the present invention may be optionally further processed as defined herein. In particular, within the scope of the present invention the fungal fermentation medium as described herein may be further processed into a dried form. To this end, the water contained in the fungal fermentation medium obtainable according to the present invention may be removed, for example by spray-drying, belt-drying, drum-drying or freeze-drying, yielding dried fungal fermentation medium, which as known to the skilled person may have improved shelf-life. Optionally, the fungal fermentation medium of the present invention may be further sterilized within the scope of the method of the present invention.

In a further embodiment, the present invention relates to use of the fungal fermentation medium of the present invention in the production of a fungal biomass. Accordingly, the fungal fermentation medium of the present invention, i.e. the medium as described hereinabove and obtainable according to the methods as disclosed hereinabove, can be used in the method of the present invention for producing a fungal biomass.

Thus, in a further embodiment, the present invention relates to a method of producing a fungal biomass, the method comprising the step of submerged fermentation of at least one fungal strain using the fungal fermentation medium of the present invention.

According to the present invention, the submerged fermentation can be operated as a batch, a fed-batch or a continuous process. These three main methods of fermentation are known to the skilled person and differ by outflow and inflow of material from/to the fermentation vessel.

As understood herein, submerged fermentation or submerged fungal fermentation is defined as cultivation of fungi in the liquid medium. As known to the skilled person, an alternative to submerged fungal fermentation is surface fungal fermentation, also referred to as solid state fungal fermentation. The liquid fungal fermentation medium, herein as understood the fungal fermentation medium of the present invention (that has been optionally pH adjusted) in solution or suspension is placed in an enclosed vessel, herein preferably a fermenter, which is usually sterilized to kill organisms that may interfere with fungal growth, according to the methods known to the skilled person. An inoculum of the at least one fungal strain as defined herein is introduced into the vessel (herein preferably fermenter) and, at least in the case of aerobic fungi, air is blown into the vessel. The contents of the vessel (fermenter herein) are preferably stirred according to the methods known to the skilled person, and preferably that can be integrated in the fermenter design. Stirring brings nutrients present in the medium and oxygen in continuous contact with the matter being fermented (herein the at least one fungal strain) and, preferably, temperature and pH are controlled at levels suitable to the fungus. After certain time, typically after between 1 to 12 days, depending on the type of fermentation, fungus, and exact fermentation conditions, among others, the fungal biomass can be harvested. (as noted by the skilled person, the timing as given herein may not necessarily apply to the cases of continuous fermentation). As however known to the skilled person, mixing may also be achieved by other methods than stirring, which may also influence the morphology of the fungal cells, as well as lead to subjecting the fungal cells to the shear stress. As understood herein, method of mixing is not meant to be limiting, and any applicable method known to the skilled person falls within the scope of the present invention.

The batch processes are characterized by lack of inflow of material into the fermentation vessel. In a batch process, all nutrients are provided at the beginning of the cultivation, without adding any more in the subsequent bioprocess. During the entire bioprocess, no additional nutrients are added with the exception of gases, acids and bases. The bioprocess then lasts until the nutrients are consumed. This strategy is suitable for rapid experiments such as strain characterization or the optimization of nutrient medium. The disadvantage of this convenient method is that the biomass and product yields are limited. Since the carbon source and/or oxygen transfer are usually the limiting factor, the microorganisms are not in the exponential growth phase for a long time. After the end of a bioprocess run in batch mode, only the biomass or medium is harvested and appropriately processed to obtain the desired product. From the bioreactor point of view, the process is repeatedly interrupted by cleaning and sterilization steps, and the biomass is only produced in stages.

In the fed batch process, substrate, nutrients and other substances may be added into the fermentation vessel, to extend the possible culture time or increase the yield, among others. The advantage of feeding during cultivation is that it allows to achieve higher product quantities overall. Under specific growth conditions, the microorganisms and/or cells constantly double and therefore follow an exponential growth curve. Therefore, in certain embodiments the feed rate may be increased exponentially as well. Generally, the substrate is pumped from the supply bottle into the culture vessel, for example through a silicone tube. The user can either manually set the feed at any time (linear, exponential, pulse-wise), or add nutrients when specific conditions are met, such as when a certain biomass concentration is reached or when a nutrient is depleted. The fed-batch process offers a wide range of control strategies and is also suitable for highly specialized applications. However, it may increase the processing time and potentially leads to inhibition through the accumulation of toxic by-products.

Preferably, in the method of the present invention the submerged fermentation is operated as a continuous process. After a batch growth phase, an equilibrium is established with respect to a particular component (also called steady state). Under these conditions, as much fresh culture medium is added, as it is removed (chemostat). These bioprocesses are referred to as continuous cultures, and are particularly suitable when an excess of nutrients would result in inhibition due to e.g. acid or ethanol build up or excessive heating. Other advantages of this method include reduced product inhibition and an improved space-time yield. When medium is removed, cells are harvested, which is why the inflow and outflow rates must be less than the doubling time of the microorganisms. Alternatively, the cells can be retained in a wide variety of ways (for example, in a spin filter), which is called perfusion. In a continuous process, the space-time yield of the bioreactor can be even further improved compared to that of a fed-batch process.

Accordingly, the fungal fermentation medium obtainable according to the method of the present invention is provided to a fermenter suitable for growing fungal mycelium. Suitable fermenters are known to the skilled person. For example, a suitable stirred tank with a specific stirrer useful in reducing the shear stress, or an airlift fermenter, is useful within the scope of the present invention. The fungal fermentation medium is understood as the medium obtainable according to the methods of the present invention and disclosed herein. As understood herein, the fungal fermentation medium can be further sterilized in certain embodiments of the present invention. As known to the skilled person, sterilization may be done by exposing the medium to elevated temperature for certain period of time. Typically, it is performed at a temperature of between 15° and 200° C. for a time of between 30 s and 10 minutes. However, the conditions as recited herein are not meant to be limiting for the scope of the present invention. For example, the process can be performed at a temperature of between 12° and 200° C., preferably 150 and 200° C., and/or for a time of between 30 s and 20 minutes, preferably for a time of between 30 s and 20 minutes.

Preferably, the fungal growth is performed at a temperature of between 15 and 40° C. Typically, a constant temperature is maintained throughout the process, which as known to the skilled person may be selected for optimal growth of a particular fungal strain. For example, in the case of P. ostreatus the growth is preferably performed at a temperature of between 25 and 30° C. Further preferably, the growth is performed at a pH of between 3.0 and 8.5. The pH of the medium can be adjusted within the scope of the method for production of the fungal fermentation medium of the present invention. As understood to the skilled person, selection of pH may be dependent on the fungal strain to be cultivated, or on potential contaminating strains to be excluded from growing. Further preferably, the growth is performed for a time of between 12 and 240 hours. As however understood to the skilled person, if the growth is performed as a continuous process, then it preferably will not be limited to 240 hours.

As understood herein, selection of the growth conditions, for example including pH, fungal fermentation medium and/or temperature, may affect the growth of the fungal mycelium, metabolism of the fungal cells, and/or whether the fungus grows as pellet or as a mycelium.

Within the scope of the method for producing a fungal biomass by submerged fermentation of at least one fungal strain of the present invention, it is understood that the at least one fungal strain is an edible fungus. Edible fungus is herein understood as a fungus that can be consumed by a mammal as food, preferably by a human, without causing any adverse reaction. Adverse reactions are herein defined as food poisoning, or undesirable taste properties that would preclude consumption. Edible fungus is herein not limited to its fruiting bodies (mushrooms), but other parts of the fungus, for example mycelium, can also be considered as an edible mushroom.

In the method for producing a fungal biomass by submerged fermentation of at least one fungal strain of the present invention, the at least one fungal strain is selected from Basidiomycota and Ascomycota.

According to the present invention, the at least one fungal strain can be selected from Basidiomycota. Preferably, the at least one fungal strain can be selected from Basidiomycota can be a fungal strain selected from Agaromycotina. As defined herein, a fungal strain selected from Agaromycotina can be a fungal strain selected from Agaricomycetes. Preferably, a fungal strain selected from Agaricomycetes can be a fungal strain selected from Boletales, Cantharellales, Agaricales, Polyporales, Russulales, and Auriculariales.

As defined herein, the fungal strain selected from Boletaceae is preferably B. edulis. In certain embodiments, a fungal strain selected from Agaricomycetes can be a fungal strain selected from Polyporales. Preferably, as defined herein, a fungal strain selected from Polyporales can be a fungal strain selected from Meripilaceae, Polyporaceae, Ganodermataceae, Sparassidaceae As defined herein, a fungal strain selected from Meripilaceae is preferably G. frondosa.

As defined herein a fungal strain selected from Polyporaceae is preferably selected from P. umbellatus and L. sulphureus. As defined herein a fungal strain selected from Sparassidaceae is preferably S. crispa.

Preferably, a fungal strain selected from Agaricomycetes can be a fungal strain selected from Cantharellales. Further preferably, a fungal strain selected from Cantharellales can be a strain selected from Cantharellaceae and Hydnaceae. As defined herein, a strain selected from Cantharellaceae can be C. cornucopioides or C. cibarius, preferably C. cibarius. As further defined herein, a strain selected from Hydnaceae can be H. repandum.

Alternatively, a fungal strain selected from Agaricomycetes can be a fungal strain selected from Boletales. Preferably, as defined herein, a fungal strain selected from Boletales can be a fungal strain selected from Boletaceae, and Sclerodermataceae.

Alternatively, a fungal strain selected from Agaricomycetes can be a fungal strain selected from Russulales. Further preferably, as defined herein, a fungal strain selected from Russulales can be a fungal strain selected from Hericiaceae, and Bondarzewiaceae. Preferably, a fungal strain selected from Russulales is a fungal strain selected from Hericiaceae, preferably selected from H. erinaceus and H. coralloides. Further preferably, the fungal strain selected from Bondarzewiaceae is B. berkeleyi.

Alternatively, a fungal strain selected from Agaricomycetes can be a fungal strain selected from Auriculariales, more preferably a fungal strain selected from Auriculariaceae. Preferably, a fungal strain selected from Auriculariaceae is A. auricula-judae.

Preferably, in the method of the present invention the at least one fungal strain is selected from Agaricales. Accordingly, the at least one fungal strain selected from Agaricales can be selected from Tuberaceae, Pleurotaceae, Agaricaceae, Marasmiaceae, Strophariaceae, Lyophyllaceae, Tricholomataceae, Omphalotaceae, Physalacriaceae, Schizophyllaceae, and Fistulinaceae.

As defined herein, the fungal strain selected Marasmiaceae from is preferably L. edodes. As further defined herein, the fungal strain selected from Strophariaceae is preferably a fungal strain selected from A. aegerita and H. capnoides. As further defined herein, the fungal strain selected from Lyophyllaceae is preferably C. Indica. As further defined herein, the fungal strain selected from Tricholomataceae is preferably a fungal strain selected from H. tesselatus and C. nuda. As further defined herein, the fungal strain selected from Omphalotaceae is preferably C. gigantean. As further defined herein, the fungal strain selected from Physalacriaceae is preferably F. velutipes. As further defined herein, the fungal strain selected from Schizophyllaceae is preferably S. commune. As further defined herein, the fungal strain selected from Fistulinaceae is preferably F. hepatica.

The at least one fungal strain according to the present invention selected from Agaricales can be selected from Tuberaceae. Preferably, the fungal strain according to the present invention selected from Tuberaceae is T. magnatum, T. estivum, T. uncinatum, T. indicum, T. rufum or T. melanosporum, more preferably T. melanosporum and T. magnatum.

More preferably, the at least one fungal strain selected from Agaricales can be a fungal strain selected from Pleurotaceae. Even more preferably, the at least one fungal strain of the present invention is a fungal strain selected from P. pulmonarius, P. ostreatus, P. citrinopileatus and P. salmoneostramineus, even more preferably selected from P. pulmonarius or P. ostreatus, most preferably P. pulmonarius.

The at least one fungal strain according to the present invention selected from Agaricales can be selected from Agaricaceae. Preferably, the fungal strain selected from Agaricaceae as defined herein is A. bisporus or A. blazei, more preferably A. bisporus.

According to the present invention, the at least one fungal strain can be selected from Ascomycota. Preferably, the at least one fungal strain can be selected from Ascomycota can be a fungal strain selected from Pezizomycotina. As defined herein, a fungal strain selected from Pezizomycotina can be selected from Pezizomycetes. Preferably, within the scope of the present invention, a fungal strain selected from Pezizomycetes can be a fungal strain selected from Pezizales.

Further preferably, the at least one fungal strain as defined in the method for production of fungal biomass of the present invention can be selected from Pezizales. Preferably, the fungal strain selected from Pezizales can be selected from Morchellaceae. Preferably, the fungal strain selected from Morchellaceae is M. esculenta, M. angusticeps or M. deliciosa.

Alternatively, the at least one fungal strain selected from Ascomycota can be a fungal strain selected from Sordariomycetes. Preferably, at least one strain as defined herein, selected from Sordariomycetes, can be a fungal strain selected from Hypocreales. Further preferably, a fungal strain selected from Hypocreales can be a fungal strain selected from Cordycipitaceae. Even further preferably, a fungal strain selected from Cordycipitaceae is a fungal strain selected from C. militaris and C. sinensis. Alternatively, a fungal strain selected from Hypocreales can be a fungal strain preferably selected from Nectriaceae. Further preferably, the fungal strain selected from Nectriaceae can be a Fusarium strain. In another embodiment, the fungal strain selected from Sordariomycetes can be a fungal strain selected from Sordariaceae. Further preferably, the fungal strain selected from Sordariaceae can be a Neurospora strain.

As disclosed herein, in the method for producing a fungal biomass by submerged fermentation of at least one fungal strain of the present invention, the at least one fungal strain can be selected from Pezizomycotina and Agaromycotina.

As further disclosed herein, in the method of the present invention for producing a fungal biomass by submerged fermentation of at least one fungal strain, the at least one fungal strain is preferably selected from Peziomycetes, Agaricomycetes and Sordariomycetes.

As further disclosed herein, in the method of the present invention for producing a fungal biomass by submerged fermentation of at least one fungal strain, the at least one fungal strain is preferably selected from Pezizales, Boletales, Cantharellales, Agaricales, Polyporales, Russulales, Auriculariales, Sordoriales and Hypocreales.

As further disclosed herein, in the method of the present invention for producing a fungal biomass by submerged fermentation of at least one fungal strain, the at least one fungal strain is preferably selected from Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, and Fistulinaceae.

As further disclosed herein, in the method of the present invention for producing a fungal biomass by submerged fermentation of at least one fungal strain, the at least one fungal strain is preferably P. pulmonarius, P. ostreatus, P. citrinopileatus or P. salmoneostramineus, even more preferably P. pulmonarius or P. ostreatus.

As further disclosed herein, in the method of the present invention for producing a fungal biomass by submerged fermentation of at least one fungal strain, the at least one fungal strain is preferably M. esculenta, M. angusticeps or M. deliciosa.

It is also encompassed within the present invention that more than one fungal strains are co-fermented, as described herein. Selection of strains for co-fermentation depends on their compatibility and can be performed by a skilled person.

It is to be understood that according to the methods of the present invention yield up to 100%, preferably up to 99% (based on conversion of carbon source available in the medium), more preferably 85 to 95%. It is understood that the biomass yield is calculated according to the ratio of the amount biomass produced to the amount of substrate(s) (e.g., C5 sugars) consumed.

In a further embodiment, the present invention relates to a fungal biomass produced according to the method for producing a fungal biomass by submerged fermentation of at least one fungal strain of the present invention. Preferably, the fungal biomass comprises the fungal cells of the fungal strain selected from Pleurotaceae, in particular wherein the fungal strain is P. pulmonarius, P. ostreatus, P. citrinopileatus or P. salmoneostramineus, more preferably selected P. pulmonarius or P. ostreatus. In another embodiment, preferably the fungal biomass comprises the fungal cells of the fungal strain selected from Morchellaceae, wherein the fungal strain is M. esculenta, M. angusticeps or M. deliciosa. However, the fungal biomass of the present invention is not limited to a single fungal strain. It is also encompassed within the present invention that more than one fungal strain are co-fermented to yield the fungal biomass of the present invention, as described herein. Selection of strains for co-fermentation depends on their compatibility and can be performed by a skilled person. Furthermore, selection of strains for co-inclusion in the fungal biomass of the present invention depends on their properties and envisaged application, as well as their growth rates, as disclosed herein.

The fungal biomass of the present invention preferably has a protein content between 10 and 60% (w/w). As further disclosed herein, the fungal biomass of the present invention preferably has a fiber content between 20 and 60% (w/w).

As indicated in Example 10, the present inventors have demonstrated that the fungal biomass of the present invention is characterized by high Asp/Asn content. Accordingly, the present invention relates to the fungal biomass of the present invention, characterized by Asp/Asn content of at least 20% of the total amino acid content, preferably at least 30% of the total amino acid content. Preferably, herein % refer to % w/w. The amino acid composition is determined preferably as described in the Examples section.

In a further embodiment, the present invention relates to use of the fungal biomass of the present invention in production of a fungal-based food product. Accordingly, the present invention also relates to a fungal-based food product, obtainable as described herein. The fungal-based food product of the present invention may be prepared in any form known to the skilled person. For example, the fungal-based food product of the present invention may take the form of a ball (i.e. meatball replacement), dumpling, vegetarian sausage, meat-replacement steak, meat-replacement ground meat product, meat-replacement product for preparing sandwiches, etc.

The food product according to the present invention may for example be a nutritional supplement. The nutritional supplement could be in the form of a liquid or a solid, such as a pill, lozenge or tablet. For example, the nutritional supplement of the present invention may be a protein supplement and/or a carbohydrate supplement.

The food product as understood herein may be a dairy product, for example yoghurt, milk drinks and ice cream. The food product as understood herein may also relate to different embodiments of seafood products, for example a crabcake, fishcake, tuna, salmon, or shrimp.

The food product may be texturized food product or a textured food product. Accordingly, the food product of the present invention comprises all amino acids necessary for human daily intake that cannot be synthetized in novo. Furthermore, the textured food product of the present invention is preferably heat-resistant, boiling resistant and suitable for cooking. For example, the fungal-based food product of the present invention, as described herein, may be a meat replacement product. It is noted that preferably the meat replacement product is a texturized food product or textured food product. It is further noted that the structure of the textured food product improves the acceptability of the textured food product by consumers. It is further noted that intrinsic fibrous texture of the fungal biomass of the present invention maybe beneficial for producing a textured food product or a texturized food product without using conventional texturizing methods such as extrusion.

According to the present inventors, the food product of the present invention is characterized by an enhanced taste. Taste properties of the food product may be determined for examples as shown in the Examples section.

As it is to be understood herein, the term “about” when referring to a temperature preferably means ±3° C., more preferably ±1° C. The term about when referring to other numerical values preferably means ±10% of said value, more preferably ±5% of said value, even more preferably ±1% of said value.

Further examples and/or embodiments are disclosed in the following numbered items.
1. A method for the production of a fungal fermentation medium from brewer's spent grain (BSG), the method comprising:
   (a1) extracting C5-sugars from the lignocellulosic material comprised in BSG via a steam pretreatment, followed by a washing step with liquid water at a temperature of not more than 50° C., and
   (b) combining the so obtained extract with at least one non-carbohydrate nutrient for fungal cultivation.
2. The method of item 1, wherein the steam pretreatment is performed at a severity factor of between 2.9 and 3.3.
3. The method of item 1 or 2, wherein during the steam pretreatment the lignocellulosic materials comprised in BSG is contacted with steam at a temperature of between 130° C. and 180° C., preferably at a temperature of between 160° C. and 180° C., more preferably at a temperature of between 165° C. and 175° C. and/or for a time up of to 30 minutes, preferably for a time of up to 15 minutes.
4. The method of any one of items 1 to 3, wherein in step (a1) washing step with liquid water is performed at a temperature of not more than 40° C., preferably not more than 30° C., more preferably not more than 25° C.
5. The method of any one of items 1 to 4, wherein the BSG is characterized by a final moisture content of between 50 wt % and 75 wt %, preferably wherein the method further comprises the step of dewatering BSG by pressing the material to a final moisture content between 50 and 75 wt %, wherein said dewatering step precedes step (a1).
6. The method of any one of items 1 to 5, wherein in step (b) the extract of step (a1) is substantially undiluted, preferably wherein said extract of step (a1) constitutes 90 to 99% v/v of the entire medium obtained in step (b), more preferably wherein said extract of step (a1) constitutes 94 to 98% v/v of the entire medium obtained in step (b).
7. The method of any one of items 1 to 6, wherein the medium is capable of supporting fungal growth without further dilution steps to reduce the concentration of fungal growth inhibitors.
8. A method for the production of a fungal fermentation medium from brewer's spent grain (BSG), the method comprising:
   (a2) extracting C5 sugars from lignocellulosic material comprised in BSG via a liquid extraction treatment with water at temperature of between 145° C. and 155° C. and/or for a time up to 70 minutes, preferably for a time up to 50 minutes, preferably at the pressure of 30 to 50 bar, and
   (b) combining the so obtained extract with at least one non-carbohydrate nutrient for fungal cultivation.
9. The method of item 8, wherein step (a2) is performed at a severity factor of between 2.9 and 3.3
10. The method of item 8 or 9, wherein the lignocellulosic material comprised in BSG is untreated, preferably wherein said material has not been dewatered.
11. The method of any one of items 1 to 10, wherein the method further comprises the step (a′) of enzymatic hydrolysis of a solid lignocellulosic residue obtained in step (a1) or in step (a2) with cellulase, separating a liquid product of hydrolysis from a solid residue, and mixing said liquid product with the extract of step (a1) or the extract of step (a2).
12. The method of any one of items 1 to 11, wherein substantially no sugar beyond that present in the extract of step (a1) or step (a2) or in the liquid product of hydrolysis of step (a′) is added.
13. The method of any one of items 1 to 12, wherein step (a1) or step (a2) is characterized by the solid load of a reactor of between 4 and 14% w/w.
14. The method of any one of items 1 to 13, wherein complex C5-polysaccharides constitute at least 50% of all sugars in the extract of step (a1) or step (a2), preferably wherein complex C5-polysaccharides constitute at least 65% of all sugars in the extract of step (a1) or step (a2), more preferably wherein complex C5-polysaccharides constitute at least 80% of all sugars the extract of step (a1) or step (a2).
15. The method of any one of items 1 to 14, wherein the medium is suitable for cultivation of at least one fungal strain selected from P. pulmonarius and M. rufobrunnea.
16. A fungal fermentation medium, obtainable according to the method of any one of items 1 to 15.
17. The fungal fermentation medium of item 16, wherein substantially all the sugar originates from BSG.
18. The fungal fermentation medium of item 16 or 17, wherein complex C5-polysaccharides constitute at least 50% of all sugars in said medium, preferably wherein complex C5-polysaccharides constitute at least 65% of all sugars in said medium, more preferably wherein complex C5-polysaccharides constitute at least 80% of all sugars in said medium said medium.
19. Use of the medium of any one of items 16 to 18 in the fungal culture.
20. A method for producing a fungal biomass, the method comprising the step of submerged fermentation of at least one fungal strain using the fungal fermentation medium of any one of items 16 to 18.
21. The method of item 20, wherein the at least one fungal strain is an edible fungus.
22. The method of item 20 or 21, wherein the at least one fungal strain is P. pulmonarius P. ostreatus, P. citrinopileatus or P. salmoneostramineus or wherein the at least one fungal strain is M. rufobrunnea, M. esculenta, M. angusticeps or M. deliciosa.
23. A fungal biomass produced according to the method of any one of items 20 to 22.
24. The fungal biomass of item 23, characterized by Asp/Asn content of at least 20% of the total amino acid content.
25. Use of the fungal biomass of item 23 or 24 in production of a food product, preferably fungal-based food product.
26. A food product, preferably a fungal-based food product, prepared using the fungal biomass of item 23 or 24.
27. The food product of claim 26, characterized by an enhanced taste.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following examples are merely illustrative of the present invention and should not be construed to limit the scope of the invention which is defined by the appended claims in any way.

### EXAMPLES

### Example 1: Steam Treatment (Extraction with Hot Water at 50° C. Or 22° C.)

Brewer's spent grain (BSG) is first treated hydrothermally under pressure to release the pentose sugars (C5 sugars) i.e., mainly xylose and arabinose. The pretreated biomass is then subjected to enzymatic hydrolysis (EH) to release glucose (C6 sugars). Hydrothermal treatment refers to a thermochemical process for decomposing biomass with water under high temperature and high-pressure conditions. Enzymatic hydrolysis (EH) or saccharification is the process of the conversion of complex carbohydrates into simpler sugars. EH was performed by using a cellulolytic enzyme. Cellulolytic enzymes break down mainly cellulose and to some extent hemicellulose into hexose (mainly glucose) and pentose (mainly xylose) sugars. The studies are carried out at different scales for the screening of efficient treatment conditions to achieve the maximum amount of sugars in the extracts with less or without toxic compounds (mycelium growth inhibitors) to provide conditions for maximum mycelium growth. In the example below, P. pulmonarius was grown on extracted sugars from BSG.

The composition of the used BSG was determined, wherein cellulose was found to be 22%, hemicellulose 25.8%, and protein 25% (based on the dry mass) having a moisture content of around 80 wt %.

To reduce the moisture content of BSG and enhance the heat transfer during steam treatment, fresh BSG with a moisture content of around 80% was treated with a hydro press at 3 bars of pressure for dewatering to a final moisture content of around 70%.

Several experiments at different process conditions were carried out to determine the most promising set of temperature and retention time. For recovery of C5 sugars, experiments of one-step treatment with steam were carried out. For the recovery of C6 sugars, a two-step steam treatment cascade was conducted prior to saccharification in order to enhance the enzymatic digestibility of BSG.

Process conditions were tested in the range of 150 to 180° C. and short retention time (5 to 20 minutes) to limit the energy consumption. Different solid loads were assessed ranging from 4 to 14% w/w.

After steam treatment, extraction was carried out in a different process unit by washing the material with water at different temperatures. According to the solid content of the material (i.e., dry matter percentage of pretreated spent grain), warm water (i.e., at 50° C.) or water around room temperature (i.e., at 22° C.) was added in a given ratio to reach the desired solid load during extraction. Constant stirring was provided for at least 20 minutes. To separate the liquid and solid fractions a hydro press was used. Liquid and solid fractions were recovered for further processing for either fermentation or further enzymatic hydrolysis, respectively.

After steam treatment, the material was collected for subsequent extraction or further steam treatment. At the end of the treatment, the material presents a homogeneous dark color suggesting that the steam diffused effectively through the bulk material.

The analysis of the samples shows that C5 sugars (xylose and arabinose) were effectively solubilized from BSG after steam treatment. Additionally, small amounts of glucose (between 1 and 3.5 g/L) were detected in the samples.

Following this value, the highest concentrations of total sugars were at treatment intensities of LogRo=3.23 and LogRo=2.94. Therefore, the best candidates for scaling up correspond to those process conditions with a severity factor ranging between 2.9 and 3.2. Additionally, the results show that at treatment intensities above (LogRo=3.5) a decrease in the sugar concentration is detected due to its thermal degradation into furfural. Furfural is a non-desired compound as it is a known potential inhibitor of mycelium growth.

Several fermentation experiments were carried out to determine the optimum conditions for mycelium growth. Before fermentation, the liquid hydrolysates were centrifuged, to reduce the quantity of fines that cause interference in the process. The screening part was performed by fermenting 50 mL of liquid hydrolysates in Erlenmeyer flasks.

The results show that the growth of the biomass, during fermentation, can be limited by some inhibitors present in the extract. Therefore, dilution of extracts for the fermentation medium is considered as an option to reduce the concentration of inhibitors and check the effect on the growth of biomass. When washing in water at 22° C., the observed mycelium growth is similar in cases where the extract is diluted or not i.e., it is proportional to the amount of extract added Accordingly, the present inventors have surprisingly found that by controlling the washing temperature the production of undesired growth inhibitors, e.g. of furfural, can be reduced. Additionally, as it is apparent to the skilled person, the inhibitors can be separated from the obtained extract using methods known to the skilled person in the art. Some of the highest concentrations of mycelium achieved are shown in the tables below.

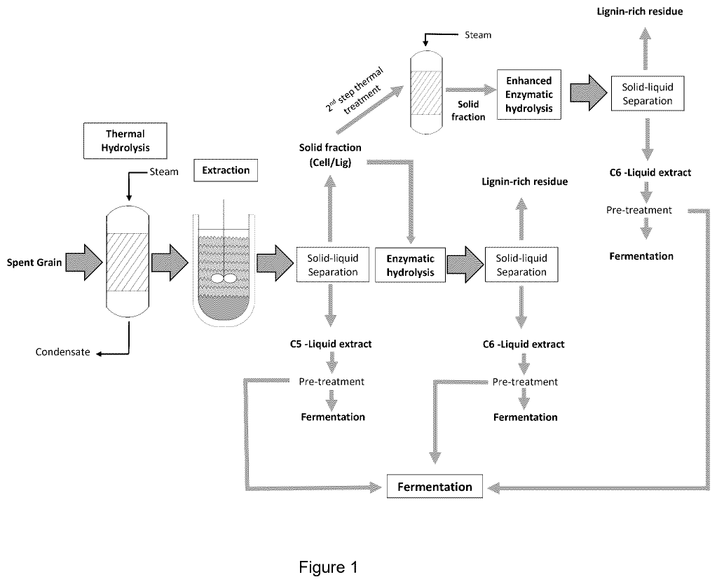

The experimental setup is shown in FIG. 1. The results are summarized in Tables 1 and 2 below.

TABLE 1.1
Experimental results of BSG steam treatment followed by extraction with water at 22 and 50° C.
in a batch set-up. Values indicated hereinbelow are related to the growth of biomass using a medium
including diluted (50%) and undiluted (100%) (a1) step extract. C5 recovery range is based on triplicates.

| | | | | | | | Biomass | | | |
|---|---|---|---|---|---|---|---|---|---|---|
| | | | | | | | [g/L per % | | | |
| Solid | Washing | Treatment | | Retention | Severity | Total | load] | | C5 | C5 |
| load | T | T | Pressure | time | factor | sugars | 50% | 100% | recovery | recovery |
| (% w/w) | (° C.) | (° C.) | (bar) | (min) | Log Ro | (g/L) | diluted | undiluted | [%] | [% range] |
| 8 | 22 | 170 | 8 | 7.5 | 2.97 | 9.8 | 0.43 | 0.86 | 34 | 30-35 |
| | 50 | 170 | 8 | 7.5 | 2.97 | 7.05 | 0.52 | 0.88 | 47 | 42-48 |
| 14 | 22 | 170 | 8 | 7.5 | 2.97 | 10.82 | 0.69 | 0.86 | 30 | 30-35 |
| | 50 | 170 | 8 | 7.5 | 2.97 | 19.2 | 0.73 | 0.95 | 53 | 50-60 |

C5 recovery as defined herein refers to the ratio of C5 sugars to the total amount of hemicellulose, determined based on the solid load of the reactor and the composition of brewer's spent grain. The maximum achieved recovery of C5 sugars is 53% (i.e., up to 60%) obtained for the condition performed at 170° C., for a retention time of 7.5 minutes, with pressure at 8 bars, solid load percentage of 14% (w/w), and the extraction with water at 50° C. The total concentration of sugars was 19.2 g/L, and the biomass concentration was 0.73 and 0.95 [g/L per % load] in 50% dilution of the extract and 100% extract used for fermentation, respectively. The effect of washing temperature at 22° C. is found to be effective for the conditions with solid load percentages 8 and 14, but not as much as to when using a washing temperature of 50° C. Therefore, this example clearly shows that a higher washing temperature is more efficient for the preparation disclosed in this invention, which was also surprising in terms of the mycelium growth using sugars extracted at a higher temperature.
Furthermore, after investigating the effect of the solid load and washing temperature on the extracts and mycelium growth, the extractor set-up was changed from batch to semi-continuous. Condition 4 in Table 1.2 shows a C5 sugar recovery up to 96% with the same conditions of Table 1.1 showing a C5 sugar recovery up to 60%. This shows the effect of the extractor configuration on the extraction yield. In the semi-continuous configuration, the feed and the reaction mixture enter and exit the system continuously, wherein the feeding and retrieving of the feedstock are not at the same speed rate, i.e., the feedstock is fed in cycles and is not stationary in the extractor as it is the case in the batch system, which increases the surface area of BSG exposed to the steam treatment. Additionally, a shredder or a disc refiner could be present or placed at the extractor outlet, wherein said shredder or said disc refiner is configured to process the brewer's spent grain so that the BSG surface area is further increased (i.e., particle sizes further reduced) for a higher sugar extraction at the washing step.

TABLE 1.2
Experimental results of BSG steam treatment with 14% (w/w) solid load followed by extraction
with hot water at 50° C. in a semi-continuous set-up. Values indicated herein below
relate to the growth of biomass using a medium including (a1) step, extract diluted by 50%.

| Conditions | 1 | 2 | 3 | 4 | 5 | 6 | 7 |
|---|---|---|---|---|---|---|---|
| Temperature (° C.) | 150 | 150 | 160 | 170 | 170 | 170 | 180 |
| Pressure (bar) | 5 | 5 | 6 | 8 | 8 | 8 | 10 |
| Retention time (min) | 7.5 | 15 | 15 | 7.5 | 15 | 30 | 10 |
| Severity factor | 2.35 | 2.65 | 2.94 | 2.94 | 3.24 | 3.54 | 3.35 |
| Total sugar [g/L per % load] | 0.72 | 0.59 | 0.92 | 2.47 | 1.94 | 0.99 | 1.26 |
| Biomass [g/L per % load] | 0.23 | 0.43 | 0.49 | 0.49 | 0.61 | 0.41 | 0.67 |
| C5 recovery [%] | 28 | 23 | 36 | 96 | 75 | 38 | 49 |

To reduce the energy requirement and cost of the process the retention time was reduced from 7.5 to 5 minutes. The BSG solid loading was 14% (w/w) for the conditions performed, as shown in Table 2 below. The results for the C5 sugar recovery, biomass growth, and furfural concentration are listed in Table 2. The recovered percentage of C5 sugars is found to be higher with a retention time of 7.5 minutes. The biomass concentration was found to be higher in the extract treated at a retention time of 7.5 minutes, in comparison to 5 minutes. No effect was observed on the furfural concentration of the change in retention time.

TABLE 2
Experimental results of BSG steam treatment with 14% (w/w) solid
load followed by extraction with hot water at 50° C. in
a batch set-up and a retention time of 5 and 7.5 minutes. Values
indicated herein below related to the growth of biomass using
a medium including (a1) step, extract diluted by 50%.

| Conditions | 1 | 2 |
|---|---|---|
| Temperature (° C.) | 170 | 170 |
| Pressure (bar) | 8 | 8 |
| Ret. Time (min) | 5 | 7.5 |
| Severity factor | 2.80 | 2.97 |
| Total sugar [g/L per % load] | 1.24 | 1.37 |
| Biomass [g/L per % load] | 0.44 | 0.73 |
| Furfural [g/L per % load] | 0.01 | 0.01 |
| C5 recovery [%] | 48% | 53% |

In a further step, to improve energy consumption and the overall economics of the process, the extraction of sugars was performed with water at three different temperatures i.e., at 22, 40, and 50° C., wherein the BSG batch was treated in a batch set-up at 170° C. for 7.5 minutes with a 14% (w/w) solid load. The concentration of C5 sugars (g/L) for the extracts obtained under the three different washing temperatures are shown in FIG. 4. The concentration of sugars decreased with decreasing temperature of the water used for the extraction. The maximum concentration of C5 sugars extracted was found for the extraction conditions with water at 50° C. (19.2 g/L) followed by 40° C. (14.5 g/L), and 22° C. (10.82 g/L). Therefore, this example clearly shows as well that a higher washing temperature is more efficient for the preparation disclosed in this invention. Depending on the desired composition of the extract, a washing temperature range between 40° C. and 50° C. is preferred, even more preferably a washing temperature range between 40° C. and 48° C. is preferred.

### Example 2: Formation of Fungal Growth Inhibitors in the Method Comprising Steam Pretreatment and Washing with Liquid Water

Furfural has proved to inhibit the growth of mycelium at concentrations as low as 0.2 g/L. This compound is a product of the thermal degradation of hemicelluloses. Even at higher furfural concentration, biomass growth can still occur due to possible effects due to other inhibitors being present (mentioned above).

The production of furfural can be partially avoided by careful control of the process conditions. In general terms, when the treatment conditions are severe, the sugars are more prone to degrade into furfural. However, even at mild treatment conditions the development of furfural cannot be completely avoided. Furfural formation is highly dependent not just on the process conditions but also on the material composition. The more free sugars the material has, the easier it is to degrade those sugars into furfural.

BSG is a material that has a composition, which is similar every time it leaves the lautering process during beer production. However, BSG is not a stable material and its composition changes fast after leaving the lautering process. In this context, the material that is delivered can contain more or less free sugars from one batch to the other. As a result, the formation of furfural is difficult to control but its appearance is much reduced when treating the BSG at rather milder conditions. The results are summarized in Table 3 below. The furfural concentration was measured by HPLC. Furfural can be also isolated or separated following known methods in the art.

The results are summarized in Table 3 below.

TABLE 3
The range of concentration of furfural observed under
different steam pretreatment and washing conditions.

| Extraction Conditions | Furfural Concentration (g/L per % load) | |
|---|---|---|
| (Table 1.1) | Washing at 22° C. | Washing at 50° C. |
| 8% solid load | 0-0.1 | 0-0.1 |
| 14% solid load | 0-0.1 | 0.1-0.3 |

### Example 3: Liquid Hot Water Treatment

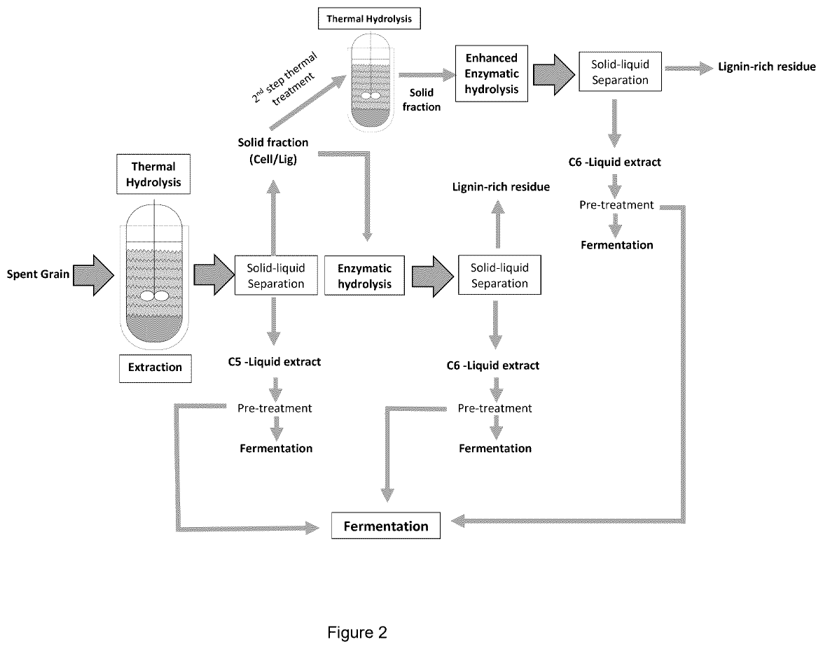

The experimental setup of this process is shown in FIG. 2. The pressure is set to be constant at 50 bar. The extraction (solubilization of C5 sugars) is performed in the same unit and the separation of liquid and solid fractions is performed with press-filtration. In this specific example, the temperature was tested in the range of 110 to 210° C. and retention times with a 10-min increment from 10 to 120 minutes to find the optimal conditions for maximum sugar extraction and maximum mycelium growth. An extract of the results is summarized in Table 4. Table 5 shows another set of conditions at a larger scale, comparing extraction results in the presence/absence of acid highlighting that using acid increases the efficiency of the liquid extraction.

TABLE 4
Experimental results of BSG liquid hot water treatment on
a small scale (30 mL extraction volume) showing the
cellular dry weight in (g) of P. pulmonarius obtained.
Temperature [° C.]

| | | 110 | 120 | 130 | 140 | 150 | 160 | 170 | 180 | 190 | 200 | 210 |
|---|---|---|---|---|---|---|---|---|---|---|---|---|
| Retention | 10 | | | 0.197 | | 0.389 | 0.495 | 0.610 | 0.341 | 0 | 0 | |
| Time | 20 | | 0.196 | | 0.289 | 0.345 | 0.536 | 0 | 0 | | | |
| [min] | 30 | | 0.207 | 0.246 | | 0.453 | 0.520 | 0 | 0 | | | |
| | 40 | 0.069 | 0.220 | 0.299 | 0.324 | 0.544 | 0.644 | 0.069 | | | | |
| | 50 | 0.159 | 0.206 | 0.408 | 0.340 | 0.712 | 0 | | | | | |
| | 60 | 0.203 | 0.208 | 0.284 | 0.464 | 0.679 | 0 | 0 | | | | |
| | 70 | | | | 0.419 | 0.397 | | | | | | |
| | 80 | | | | | 0.118 | | | | | | |
| | 90 | | 0.250 | | 0.170 | 0.544 | | | | | | |
| | 120 | 0.186 | | 0.369 | | | 0 | | | | | 0 |

TABLE 5
An extract of some experimental results of BSG liquid hot water treatment on a larger
scale. Values indicated hereinbelow related to the growth of biomass using a medium including undiluted
(a2) step extract. In Conditions 5, 7, 0.92% w/w of H₂SO₄ was added and in condition 6, 0.5% w/w of
H₂SO₄ was added. If condition 6 is modified to have the same acid concentration compared
to 5 and 7, the sugar recovery obtained is 82%.

| Conditions | 1 | 2 | 3 | 4 | 5 | 6 | 7 |
|---|---|---|---|---|---|---|---|
| Temperature (° C.) | 110 | 140 | 150 | 180 | 140 | 145 | 150 |
| Pressure (bar) | 50 | 50 | 50 | 50 | 50 | 50 | 50 |
| Ret. Time (min) | 60 | 60 | 50 | 20 | 5 | 5 | 5 |
| Severity factor | 2.07 | 2.96 | 3.17 | 3.66 | 1.88 | 2.02 | 2.17 |
| Total sugar [g/L per % load] | 0.575 | 1.2125 | 1.575 | 2.3125 | 2.84 | 2.83 | 3.46 |
| Biomass [g/L per % load] | 0.5075 | 1.16 | 1.78 | 0 | 1.08 | 0.97 | 1.45 |
| Furfural [g/L per % load] | 0 | 0 | 0 | 0.07 | 0.04 | 0.08 | 0.08 |
| C5 recovery [%] | 7.585 | 19.275 | 29.07 | 45.8 | 77 | 73 | 92 |

### Example 4: Contamination Test

All the chemicals required for media preparation were purchased from Carl Roth (Karlsruhe, Germany), VWR (Darmstadt, Germany), Merck KgaA (Darmstadt, Germany) or Sigma-Aldrich (Steinheim, Germany).

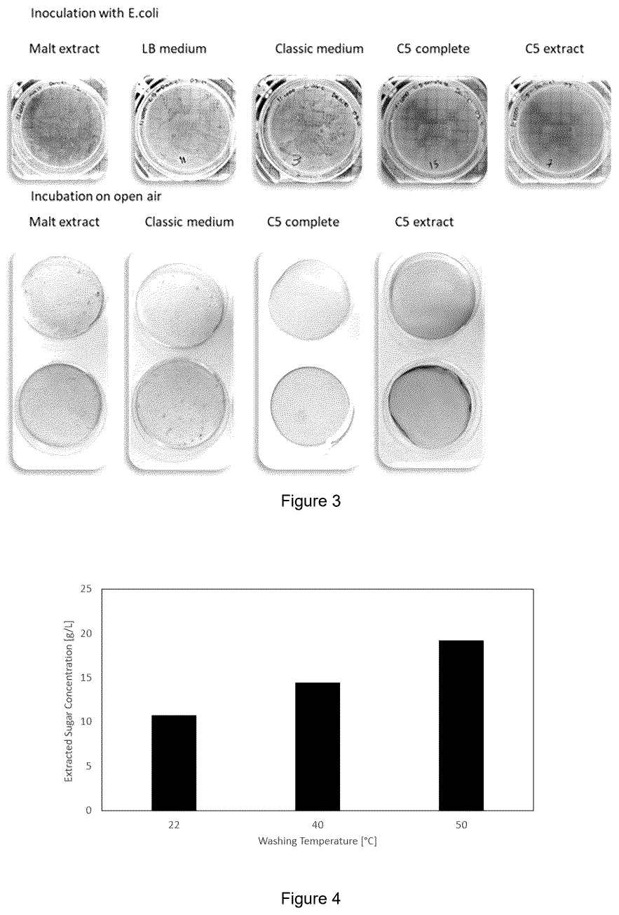

The media for preparation of the agar plate were prepared by weighing the different compounds according to Table 6 and dissolving them in water afterward. The media according to the present invention described as C5 extract were prepared as in Example 6 condition two. C5 complete refers to a C5 extract as described herein, further supplemented with 5.9 g/I K₂HPO₄, 9.0 g/L KH₂PO₄, 12.0·10⁻² g/L MgSO₄, 8.10·10⁻³ FeCl₃, and 10.10·10⁻³ CaCl₂. The mixtures were then autoclaved for 20 min at 121° C. and subsequently poured aseptically under a sterile clean bench and left there until complete solidification. Subsequently, the plates prepared with LB medium, malt extract, M9 classic medium, C5 extract, and C5 complete were inoculated with a 100 μL 1:1000 diluted E. coli suspension obtained from a culture in LB medium incubated at 37° C. overnight. The plates were then incubated at 37° C. overnight and photographed on the next day. In another experiment, the lid of the plates prepared with malt extract, M9 classic medium, C5 extract and C5 complete were just removed and exposed to air contamination to compare growth on the different media. In that case, pictures of the plates were made after 10 days (see FIG. 3 for summary).

TABLE 6
Composition of media used for the contamination tests.

| | |
|---|---|
| Compound | Concentration [g L⁻¹] |

LB medium

| | |
|---|---|
| NaCl | 10 |
| Tryptone | 10 |
| Yeast extract | 5 |
| Agar | 20 |

Fungal fermentation medium

| | |
|---|---|
| Malt extract | 30 |
| Peptone | 5 |
| Agar | 20 |

M9 “Classic medium”

| | |
|---|---|
| Glucose | 10.0 |
| KH₂PO₄ | 3.00 |
| NH₄Cl | 1.50 |
| Na₂HPO₄ | 6.70 |
| FeCl₃ | 8.10 · 10⁻³ |
| CaCl₂ | 10.0 · 10⁻³ |
| MgSO₄ | 12.0 · 10⁻² |
| ZnCl₂ | 17.0 · 10⁻⁴ |
| MnCl₂ | 10.0 · 10⁻⁴ |
| NaMoO₄•2H2O | 60.0 · 10⁻⁵ |
| CoCl₂ | 32.8 · 10⁻⁵ |
| CuCl₂•2H2O | 43.0 · 10⁻⁵ |
| NaCl | 10.0 |
| Agar | 20.0 |

### Example 5: Production of a Fungal Fermentation Medium from Spent Grain Using Liquid Extraction with Added Acid

Extraction experiments using liquid water were performed on spent grain according to the general protocol described hereinabove or in Table 5, wherein of H₂SO₄ were added to the water used for extraction at different concentrations. The experiments were performed according to the conditions as discussed in Table 7.

TABLE 7
Summary of the extraction conditions
of spent grain with diluted acid.

Treatment Parameters

| Experiment | Temperature | Time | Severity | H2SO4 |
|---|---|---|---|---|
| [—] | [° C.] | [min] | log (Ro) | [% w/w] |
| 1 | 110 | 20 | 1.59 | 0.4 |
| 2 | 110 | 20 | 1.59 | 0.7 |
| 3 | 130 | 40 | 2.48 | 0.7 |
| 4 | 140 | 30 | 2.65 | 0.4 |
| 5 | 140 | 60 | 2.95 | 0.7 |
| 6 | 160 | 10 | 2.76 | 0.7 |
| 7 | 145 | 5 | 2.02 | 0.5 |
| 8 | 140 | 10 | 2.18 | 0.7 |
| 9 | 130 | 5 | 1.58 | 1.5 |

Similarly to the previous examples, the protocol “Analysis of sugar and furfural content” was used to quantify recovery of C5 sugars and furfural concentration and fermentation was performed on the obtained extracts afterwards using the protocol “Cultivation of fungal biomass”. The outcome of both extraction and fermentation are summarized in Table 8.

TABLE 8
Summary of the extraction experiments
with spent grain using diluted acid.

| Condition | Furfural | C5 sugars - | Biomass on C5 |
|---|---|---|---|
| (Table 7) | [g/L per % load] | Recovery [%] | [g/L per % load] |
| 1 | 0.000 | 34.64 | 2.055 |
| 2 | 0.000 | 47.20 | 0.7825 |
| 3 | 0.000 | 72.31 | 1.865 |
| 4 | 0.000 | 62.35 | 1.30 |
| 5 | 0.175 | 96.12 | n.d. |
| 6 | 0.000 | 97.42 | n.d. |
| 7 | 0.08 | 73 | 0.97 after 50% dilution |
| 8 | 0.15 | 82 | 1.11 after 50% dilution |
| 9 | 0.04 | 88 | 1.11 after 50% dilution |

### Example 6. Hydrolysis of Cellulose from Pretreated Spent Grain

Extraction experiments were performed on spent grain using ten different conditions as defined in Table 9 below, according to the general protocol as outlined in the section “Thermal extraction of the lignocellulosic material” and biomass was subsequently produced using the protocol “Cultivation of fungal biomass”.

### Thermal Extraction of the Lignocellulosic Material

For thermal extraction, the dry matter of the lignocellulosic material was determined with a moisture analyser (160° C. until constant weight) using 3 g of the material. Based on the obtained dry matter, the reactor load was adjusted by weighing the required amount of material and introducing it to the reactor afterwards. Water was then added to fill up the reactor to its final working volume and a rubber sealing ring was placed on top of the reactor housing. The lid which is equipped with a manometer and temperature probe was then tightly closed to avoid leakages and the reactor was then pressurized with nitrogen until the desired operation pressure is reached. After that, the desired temperature, pressure, and retention time were set in the software and extraction was started. During the experiment, temperature and pressure were constantly monitored and regulated.

At the end of the extraction, the reactor was cooled down by submerging it in cool water or, for larger reactors, using the cooling jacket. Once the temperature decreased below 30° C., the reactor was depressurized by opening slowly the exhaust valve and the lid opened as soon as the pressure reaches atmospheric pressure. In the case of a continuous extraction process, the mixture was cooled after extraction from the reactor using a heat exchanger. The solid and liquid phase (extract) were then separated either by press filtration or with a decanter centrifuge. The liquid extracts were then either shortly stored at 4° C. or freeze at −20° C. for long time storage. The remaining treated solid lignocellulosic material was further submitted to enzymatic hydrolysis to recover glucose from cellulose.

### Cultivation of Fungal Biomass

The liquid extracts obtained after thermal extraction were either used directly as a growth medium, mixed and/or supplemented with additional compounds (5.9 g/l K₂HPO₄, 9.0 g/L KH₂PO₄, 12.0·10⁻² g/L MgSO₄, 8.10·10⁻³ FeCl₃, 10.10·10⁻³ CaCl₂) and other trace elements according to typical M9 medium (Miller, 1972, Experiments in Molecular Genetics. Cold Spring Harbor, NY: New York Cold Spring Harbor Laboratory). The obtained mixture was then placed in appropriate vessel and sterilised (121° C., 20 min). After sterilisation, the medium was let to cool to room temperature and inoculated with spores or mycelium from a suspension prepared from a fully grown mycelium agar plate. The broth was then incubated at a temperature of 30° C. for a time of 5 days. The pH was regulated using acid and base during fermentation to keep it at the optimal pH of 6.5 for the cultivated strain of P. pulmonarius. After that, the biomass was harvested by centrifugation, washed with water and the dry matter finally determined using a moisture analyser (160° C. until constant weight). The dry matter was converted to a biomass concentration using the broth volume.

The reference biomass used for comparison with biomass produced from extracts was produced using a reference medium consisting of glucose, a trace solution containing magnesium, iron, manganese, zinc, copper and calcium as well as yeast extract as nitrogen source. The pH of the medium was adjusted to 6.5 using phosphate buffer and flasks were incubated at 30° C. for 5 days.

TABLE 9
Summary of the extraction conditions for spent grain.

| | Treatment Parameters | | |
|---|---|---|---|
| Experiment | Temperature | Time | Severity |
| [—] | [° C.] | [min] | log (Ro) |
| 1 | 180 | 20 | 3.65652549 |
| 2 | 150 | 50 | 3.171 |
| 3 | 150 | 90 | 3.426 |
| 4 | 170 | 10 | 3.061 |
| 5 | 200 | 10 | 3.944 |
| 6 | 120 | 30 | 2.066 |
| 7 | 140 | 20 | 2.479 |
| 8 | 150 | 20 | 2.773 |

The solid lignocellulosic materials recovered after thermal extraction of spent grain of Example 1, herein the spent grain, was treated according to the methods as described in the section “Hydrolysis of lignocellulosic material”. Recovery of C6-polysaccharides fraction determined as described in “Analysis of sugar and furfural content” as well as the biomass obtained (expressed as per % solid loaded for comparison purposes) from fermentation trials with these extracts performed as described in the protocol “cultivation of fungal biomass” are summarized in Table 10.

### Hydrolysis of the Lignocellulosic Material

The dry matter of the recovered lignocellulosic material after treatment as described in the section “Thermal extraction of the lignocellulosic material” was determined using a moisture analyser (incubation at 160° C. until constant weight) and the load was adjusted as described herein for the thermal treatment. The material was then loaded into the reaction vessel and the enzyme cocktail (Ctec2 and Ctec3 from Novozymes) added according to the cellulose content in the material and following the manufacturer's guidelines. The mixture of enzyme and cellulosic material was then incubated for a time between 10 and 200 hours at a temperature of 60° C. and a pH of 6.0. The homogeneity of the mixture was ensured either by a stirrer or by incubating the vessel in an incubator. After the incubation, the reaction mixture was quickly cooled using either an ice bath, the cooling jacket of the reaction vessel or in case of a continuous process, a heat exchanger. Finally, the liquid and solid fractions were separated by press filtration or centrifugation. The solid fraction rich in lignin was stored at 4° C. before analysis and the cleared liquid fraction used for fermentation experiments.

TABLE 10
Summary of hydrolysis experiments
performed on the pretreated BSG.

| | C6 sugar - | C6 sugar recovery | Biomass on C6 |
|---|---|---|---|
| Condition | Recovery [%] | [g/L per % load] | [g/L per % load] |
| 1 | 100 | 3.86475 | n.d |
| 2 | 72.6 | 2.45175 | 1.17 |
| 3 | 89.94171891 | 3.123142857 | 1.74 |
| 4 | 96.5 | 3.018571429 | 2.05 |
| 5 | 86.7 | 3.738285714 | n.d |
| 6 | 91.51375086 | 2.315555556 | 1.50 |
| 7 | 67.44284851 | 2.081 | 0.68 |
| 8 | 75.11933099 | 2.431 | 1.41 |

### Example 7: Production of a Fungal Fermentation Medium from Spent Grain Using Steam Prehydrolysis

Extraction experiments with steam prehydrolysis were performed on the spent grain as described in protocol “Thermal pretreatment and extraction of lignocellulosic biomass using steam”.

### Thermal Pretreatment and Extraction of Lignocellulosic Biomass Using Steam

Prior to operation, the reactor is preheated with steam until the operational temperature is reached. During the preheating phase the condensation, resulting from the steam getting in contact with the cold reactor, must be removed.

Material is weighted and loaded in the reactor either in a metal cartridge (batch treatment) or using a screw feeder (continuous treatment). Batch extraction is performed in a closed system, wherein the spent grain and water is loaded just once at the beginning in the reactors. Once the reactors are pressurized the system remains closed until the reaction mixture is cooled down to at least 40° C. Whereas continuous extraction refers to the continuous feeding and retrieving of the feedstock at the same speed rate. When the material is fed there is no pressure drop inside the reactor and when the material is retrieved, a sudden drop in pressure is created resulting in a steam explosion. However, in a semi-continuous process, the feed and the reaction mixture enter and exit the system continuously. The feeding and retrieving of the feedstock are not at the same rate, as the feedstock is fed in cycles.

Then, the material is pretreated, for the desired residence time with a constant injection of steam, the temperature inside the reactor is controlled by setting the pressure controller to the steam pressure corresponding to the desired temperature. In the case of continuous treatment, the residence time is controlled with the rotation speed of the screw conveyor reactor.

After the residence time is achieved, the material is recovered from the reactor, its weight and moisture content are recorded to further continue with the next treatment process (extraction).

Extraction is performed in a separate unit, steam-pretreated material is mixed with warm water (between 4° and 60° C., preferably between 4° and 50° C.) in the desired ratio to achieve a certain solid load. Constant mixing is provided by a stirred tank for at least around 20 minutes before the liquid and solid fractions are separated.

The solid-liquid mixture is pumped to a pressing machine where the two fractions are separated at a constant pressure of around 4 bar. The liquid hydrolysate (rich is C5 sugars) as well as the solid fraction (rich in cellulose and lignin) are recovered for further treatment.

The solid fraction can then be further treated with enzymes for the conversion of cellulose into C6 sugars.

### Analysis of Sugars and Furfural Content

For C5 extracts, i.e., as obtainable according to the section “Thermal extraction of the lignocellulosic material”, the samples were hydrolysed with hydrochloric acid (final concentration 4% w/w) at 121° C. for 60 min before analysis to be able to quantify the recovered C5 sugars as monosaccharides. After hydrolysis, the pH of the extract was then set to 5.5 to avoid damaging the HPLC column. The monosaccharides from C6 extracts, as obtainable according to the section “Hydrolysis of the lignocellulosic material and from C5 extracts as described above, were then measured with an Agilent HPLC 1200 system using a Metacarb 87C column (300×7.8 mm, Varian Inc, Paolo Alto, CA, USA) as the stationary and ultrapure water as the mobile phase. The measurement was performed at a temperature of 85° C. and an isocratic flow of 0.6 mL min⁻¹. After column separation, the analytes were detected by a refractive index detector, except in the case of furfural that was measured using a UV-detector at a wavelength of 270 nm.

The experimental conditions for the prehydrolysis are listed in Table 11. The results of sugar and furfural analysis (Protocol “Analysis of sugar and furfural content”) as well as the biomass (expressed as per % solid sidestream loaded for comparison purposes) obtained from fermentations with the resulting extracts (Protocol “Cultivation of fungal biomass”) are presented in Table 12.

TABLE 11
Summary of the extraction conditions of
spent grain using steam prehydrolysis.

| | Treatment Parameters | | |
|---|---|---|---|
| Experiment | Temperature | Time | Severity |
| [—] | [° C.] | [min] | log (Ro) |
| 1 | 150 | 7.5 | 2.35 |
| 2 | 150 | 15 | 2.65 |
| 3 | 160 | 7.5 | 2.64 |
| 4 | 160 | 15 | 2.94 |
| 5 | 170 | 7.5 | 2.94 |
| 6 | 170 | 15 | 3.24 |
| 7 | 170 | 30 | 3.54 |
| 8 | 180 | 7.5 | 3.23 |
| 9 | 180 | 10 | 3.36 |
| 10 | 180 | 20 | 3.66 |

TABLE 12
Summary of the extraction experiments with
spent grain using steam prehydrolysis.

| Condition | C5 sugars - | Biomass on C5 | Complex |
|---|---|---|---|
| (Table 11) | Recovery [%] | [g/L per % load] | C5 [%] |
| 1 | 20.59 | 0.47 | 88.02 |
| 2 | 16.75 | 0.86 | 84.22 |
| 3 | 26.93 | 0.69 | 87.41 |
| 4 | 26.25 | 0.97 | 87.60 |
| 5 | 72.41 | 1.05 | 88.15 |
| 6 | 52.95 | 1.23 | 89.34 |
| 7 | 28.96 | 0.82 | 92.03 |
| 8 | 51.37 | 0.57 | n.d. |
| 9 | 39.83 | 1.34 | n.d. |
| 10 | 36.39 | 0 | n.d. |

### Example 8—Hydrolysis of Cellulose from Pretreated Spent Rain from Example 7

The pretreated solid material left after the steam prehydrolysis and recovery with water was recovered, subjected to a second prehydrolysis and finally to an enzymatic hydrolysis as described in “Hydrolysis of the lignocellulosic material”. The conditions tested are listed in Table 13.

TABLE 13
Summary of the extraction conditions of pretreated
spent grain using steam prehydrolysis.

Treatment Parameters

| Experiment | Temperature | Time | Severity |
|---|---|---|---|
| [—] | [° C.] | [min] | log (Ro) |
| 1 | 150 | 15 | 2.65 |
| 2 | 160 | 7.5 | 2.64 |
| 3 | 160 | 15 | 2.94 |
| 4 | 170 | 7.5 | 2.94 |
| 5 | 170 | 30 | 3.54 |
| 6 | 170 | 7.5 + 15 | 2.94 + 3.237 |
| 7 | 170 | 7.5 + 30 | 2.94 + 3.538 |
| 8 | 170 | 15 + 30 | 3.237 + 3.538 |

The data on the recovery of C6 sugars as well as obtainable biomass (measurements performed as in the general protocols “Analysis of sugar and furfural content” and “Cultivation of fungal biomass” included hereinabove) are summarized in Table 14. For comparison purposes, they are normalized to the solid load used for the experiments.

TABLE 14
Summary of the extraction experiments with
spent grain using steam prehydrolysis.

| Condition | C6 sugars - Recovery | Biomass on C6 |
|---|---|---|
| (Table 13) | [g/L per % load] | [g/L per % load] |
| 1 | 1.66 | n.d. |
| 2 | 1.91 | 1.67 |
| 3 | 2.23 | 1.41 |
| 4 | 1.77 | 1.34 |
| 5 | 2.00 | 1.29 |
| 6 | 2.69 | 1.46 |
| 7 | 2.99 | 1.80 |
| 8 | 3.31 | 1.77 |

### Example 9. Sensory Evaluation of Meatballs Produced with Mycelium from Different Origins

Meatballs are formed from mycelium biomass and fried in a pan. Each trained panellist is blindfolded and successively receives a meatball prepared with mycelium either from the standard medium and one from spent grain. During this first session, they define the sensory attributes they recognise in the two meatballs. Subsequently, they discuss the attributes together and choose common attributes that every panellist can associate to the same taste and aroma of the meatballs to compare them. A second session is then started, and the panellists have to evaluate the meatballs according to the chosen attributes and put a score between 0 and 5 for each attribute. This session is repeated on different days to increase statistical relevance of data and average of scoring is calculated and plotted on a spider web.

The colour is determined using the RGB system and a colour analyser at 20 different positions on the meatballs. The positions used for the 10 measurements are the same for all meatballs. The mean values of these measurements are then used to compare the colour of the meatballs.

### Example 10. Amino Acid Content Analysis of Obtained Biomass

The amino acid content of the biomass obtained as described in Table 9 using a medium obtained in a process of extracting brewer's spent grain with liquid water has been determined as follows.

The amino acid profile of proteins extracted from the lignocellulosic material was determined by hydrolysing samples of extracts at 105° C. for 24 h with 6 M HCl prior to HPLC measurement. Hydrolysates were subsequently evaporated under a nitrogen stream and resuspended in 200 μM α-aminobutyrate, the latter serving as internal standard. Amino acid concentrations in the prepared samples were finally measured by fluorescence detection using an Agilent 1200 HPLC system (Agilent technologies, Waldbronn, Germany) equipped with a reverse phase column Gemini 5p C18 110 A (150×4.6 mm, Phenomenex, Aschaffenburg, Germany) as stationary phase. Separation of the different proteinogenic amino acids relied on a gradual change of the mobile phase composition throughout the measurement, mixing differently eluent A (40 mM NaH2PO4, pH 7.8) and eluent B (45% methanol, 45% acetonitrile, 10% water) according to a well-defined gradient profile. Moreover, column separation was operated at 40° C. with a flow rate of 1 mL min⁻¹. In addition, a pre-column (Gemini C18, MAX, RP, 4×3 mm, Phenomenex, Aschaffenburg, Germany) was used to increase column lifetime. Fluorescence detection was achieved through pre-column derivatisation with o-phtalaldehyde (OPA) and 9-fluorenylmethyloxycarbonyl (FMOC) and modification of the excitation and emission wavelength (Table 15).

TABLE 15
Method used for separation and quantification of amino
acids - Composition of the mobile phase was varied
during measurement to achieved separation by gradient elution. Eluent
A: 40 mM NaH₂PO₄, pH 7.8; Eluent B: 45% methanol,
45% acetonitrile, 10% water.

| Time | Eluent A | Eluent B | Excitation λ | Emission λ |
|---|---|---|---|---|
| [min] | [%] | [%] | [nm] | [nm] |
| 0 | 100 | 0.0 | 340 | 450 |
| 40.5 | 59.5 | 40.5 | 340 | 450 |
| 41 | 39 | 61 | 340 | 450 |
| 43 | 39 | 61 | 266 | 305 |
| 57.5 | 0.0 | 100 | 266 | 305 |
| 59.5 | 0.0 | 100 | 340 | 450 |
| 60.5 | 25.0 | 75.0 | 340 | 450 |
| 61.5 | 50.0 | 50.0 | 340 | 450 |
| 62.5 | 75.0 | 25.0 | 340 | 450 |
| 63.5 | 100.0 | 0.0 | 340 | 450 |
| 65.5 | 100.0 | 0.0 | 340 | 450 |

The results are shown in Table 16 hereinbelow.

TABLE 16
Amino acid content of the obtained biomass, expressed in
% w/w (YE—yeast extract, CSL—corn steep liquor).

| AA | YE | CSL | 1 | 2 | 3 |
|---|---|---|---|---|---|
| Ala | 9.06% | 13.32% | 6.2% | 5.4% | 5.6% |
| Gly | 4.94% | 7.84% | 11.6% | 10.2% | 10.4% |
| Ile | 5.27% | 4.23% | 0.0% | 5.8% | 1.5% |
| Leu | 7.58% | 11.29% | 2.0% | 3.7% | 2.9% |
| Val | 6.26% | 7.21% | 2.7% | 3.2% | 2.9% |
| Phe | 4.45% | 4.70% | 1.3% | 2.5% | 1.7% |
| Tyr | 3.62% | 1.41% | 1.3% | 2.4% | 11.3% |
| Asp/Asn | 10.05% | 8.31% | 63.8% | 39.0% | 44.0% |
| Glu/Gln | 16.31% | 19.75% | 1.3% | 1.9% | 1.4% |
| Arg | 3.46% | 2.35% | 3.1% | 3.5% | 2.3% |
| His | 2.31% | 3.13% | 0.8% | 1.6% | 1.3% |
| Lys | 8.40% | 2.51% | 0.8% | 3.1% | 1.6% |
| Ser | 4.94% | 5.80% | 3.7% | 3.4% | 3.7% |
| Thr | 4.94% | 5.49% | 1.5% | 6.0% | 1.6% |
| Met | 8.40% | 2.66% | 0.0% | 8.3% | 7.9% |
| AA | 4 | 5 | 6 | 7 | 8 |
| Ala | 6.2% | 5.5% | 21.9% | 13.4% | 14.7% |
| Gly | 10.7% | 13.6% | 8.5% | 10.3% | 10.7% |
| Ile | 2.4% | 1.4% | 0.0% | 1.6% | 0.0% |
| Leu | 2.8% | 2.0% | 2.3% | 3.1% | 2.6% |
| Val | 3.6% | 3.3% | 2.9% | 4.9% | 2.8% |
| Phe | 1.7% | 1.2% | 4.6% | 9.3% | 2.9% |
| Tyr | 12.3% | 11.3% | 3.2% | 1.5% | 2.6% |
| Asp/Asn | 39.4% | 39.9% | 7.1% | 22.7% | 35.0% |
| Glu/Gln | 1.8% | 2.1% | 22.0% | 7.2% | 6.1% |
| Arg | 2.3% | 2.7% | 4.9% | 5.7% | 4.2% |
| His | 1.6% | 0.9% | 4.1% | 1.5% | 3.2% |
| Lys | 2.4% | 0.0% | 14.1% | 6.6% | 10.0% |
| Ser | 3.2% | 3.4% | 3.3% | 4.0% | 3.9% |
| Thr | 1.5% | 1.4% | 1.2% | 0.0% | 1.3% |
| Met | 8.0% | 11.4% | 0.0% | 8.1% | 0.0% |

### Example 11: Production of a Fungal Fermentation Medium from Wheat Bran

Extraction experiments were performed on wheat bran using 4 different conditions as defined in Table 24 below, according to the general protocol as outlined in the section “Thermal extraction of the lignocellulosic material”. The pH of the water was adjusted to around 13.5 using NaOH to extract the sugars from wheat bran reaching at least 80% sugar recovery.

TABLE 17
Summary of the extraction conditions of wheat
bran and related fermentation outcome

| | Treatment parameters | | | Biomass on C5 |
|---|---|---|---|---|
| Experiment | Temperature | Time | Severity | extracts |
| [—] | [° C.] | [min] | log (Ro) | [g/L per % load] |
| 1 | 120 | 20 | 1.89 | 2.2632 |
| 2 | 110 | 10 | 1.294 | 0.4555 |
| 3 | 130 | 10 | 1.883 | 1.26775 |
| 4 | 150 | 10 | 2.472 | 0.91725 |

Further examples and/or embodiments of the present invention are disclosed in the following numbered clauses.
1. A method for the production of a fungal fermentation medium from brewer's spent grain (BSG).
2 The method of clause 1, the method comprising:
   (a1) extracting C5-sugars from the lignocellulosic material comprised in BSG via a steam pretreatment, followed by a washing step with liquid water at a temperature of not more than 50° C., and
   (b) combining the so obtained extract with at least one non-carbohydrate nutrient for fungal cultivation.
3. The method of clause 2, wherein during the steam pretreatment the lignocellulosic materials comprised in BSG is contacted with steam at a temperature of between 130° C. and 180° C., preferably at a temperature of between 160° C. and 180° C., more preferably at a temperature of between 165° C. and 175° C. and/or for a time up of to 30 minutes, preferably for a time of up to 15 minutes, and/or
   wherein in step (a1) washing step with liquid water is performed at a temperature of not more than 40° C., preferably not more than 30° C., more preferably not more than 25° C.
4. The method of clause 2 or 3, wherein the BSG is characterized by a final moisture content of between 50 wt % and 75 wt %, preferably wherein the method further comprises the step of dewatering BSG by pressing the material to a final moisture content between 50 and 75 wt %, wherein said dewatering step precedes step (a1), and/or
   wherein in step (b) the extract of step (a1) is substantially undiluted, preferably wherein said extract of step (a1) constitutes 90 to 99% v/v of the entire medium obtained in step (b), more preferably wherein said extract of step (a1) constitutes 94 to 98% v/v of the entire medium obtained in step (b), and/or wherein the medium is capable of supporting fungal growth without further dilution steps to reduce the concentration of fungal growth inhibitors.
5. The method of clause 1, the method comprising:
   (a2) extracting C5 sugars from lignocellulosic material comprised in BSG via a liquid extraction treatment with water at temperature of between 145° C. and 155° C. and/or for a time up to 70 minutes, preferably for a time up to 50 minutes, preferably at the pressure of 30 to 50 bar, and
   (b) combining the so obtained extract with at least one non-carbohydrate nutrient for fungal cultivation,
   preferably wherein the lignocellulosic material comprised in BSG is untreated, more preferably wherein said material has not been dewatered.
6. The method of any one of clauses 2 to 5, wherein step (a1) or step (a2) is performed at a severity factor of between 2.9 and 3.3.
7. The method of any one of clauses 1 to 6, wherein the method further comprises the step (a′) of enzymatic hydrolysis of a solid lignocellulosic residue obtained in step (a1) or in step (a2) with cellulase, separating a liquid product of hydrolysis from a solid residue, and mixing said liquid product with the extract of step (a1) or the extract of step (a2).
8. The method of any one of clauses 1 to 7, wherein substantially no sugar beyond that present in the extract of step (a1) or step (a2) or in the liquid product of hydrolysis of step (a′) is added, and/or
   wherein step (a1) or step (a2) is characterized by the solid load of a reactor of between 4 and 14% w/w, and/or
   wherein complex C5-polysaccharides constitute at least 50% of all sugars in the extract of step (a1) or step (a2), preferably wherein complex C5-polysaccharides constitute at least 65% of all sugars in the extract of step (a1) or step (a2), more preferably wherein complex C5-polysaccharides constitute at least 80% of all sugars the extract of step (a1) or step (a2), and/or wherein the medium is suitable for cultivation of at least one fungal strain selected from P. pulmonarius and M. rufobrunnea.
9. A fungal fermentation medium, obtainable according to the method of any one of clauses 1 to 8.
10. The fungal fermentation medium of clause 9, wherein substantially all the sugar originates from BSG, and/or
   wherein complex C5-polysaccharides constitute at least 50% of all sugars in said medium, preferably wherein complex C5-polysaccharides constitute at least 65% of all sugars in said medium, more preferably wherein complex C5-polysaccharides constitute at least 80% of all sugars in said medium said medium.
11. Use of the medium of clause 9 or 10 in the fungal culture.
12. A method for producing a fungal biomass, the method comprising the step of submerged fermentation of at least one fungal strain using the fungal fermentation medium of clause 9 or 10, preferably wherein the at least one fungal strain is an edible fungus, more preferably wherein the at least one fungal strain is P. pulmonarius P. ostreatus, P. citrinopileatus or P. salmoneostramineus or wherein the at least one fungal strain is M. rufobrunnea, M. esculenta, M. angusticeps or M. deliciosa.
13. A fungal biomass produced according to the method of clause 12, preferably characterized by Asp/Asn content of at least 20% of the total amino acid content.
14. Use of the fungal biomass of clause 13 in production of a food product, preferably fungal-based food product.
15. A food product, preferably a fungal-based food product, prepared using the fungal biomass of clause 14.

1. A method for the production of a fungal fermentation medium from brewer's spent grain (BSG).

2. The method of claim 1, the method comprising:
(a1) extracting C5-sugars from the lignocellulosic material comprised in BSG via a steam pretreatment, followed by a washing step with liquid water at a temperature of not more than 50° C., and
(b) combining the so obtained extract with at least one non-carbohydrate nutrient for fungal cultivation.

3. The method of claim 2, wherein during the steam pretreatment the lignocellulosic materials comprised in BSG is contacted with steam at a temperature of between 130° C. and 180° C.

4. The method of claim 3, wherein during the steam pretreatment the lignocellulosic materials comprised in BSG is contacted with steam at a temperature of between 160° C. and 180° C.

5. The method of claim 3 or 4, wherein during the steam pretreatment the lignocellulosic materials comprised in BSG is contacted with steam at a temperature of between 165° C. and 175° C.

6. The method of any one of claims 3 to 5, wherein during the steam pretreatment the lignocellulosic materials comprised in BSG is contacted with steam for a time up of to 30 minutes.

7. The method of any one of claims 3 to 6, wherein during the steam pretreatment the lignocellulosic materials comprised in BSG is contacted with steam for a time of up to 15 minutes.

8. The method of any one of claims 3 to 7, wherein in step (a1) washing step with liquid water is performed at a temperature of not more than 40° C., preferably not more than 30° C., more preferably not more than 25° C.

9. The method of any one of claims claim 2 to 8, wherein the BSG is characterized by a final moisture content of between 50 wt % and 75 wt %.

10. The method of claim 9, wherein the method further comprises the step of dewatering BSG by pressing the material to a final moisture content between 50 and 75 wt %, wherein said dewatering step precedes step (a1).

11. The method of claim 9 or 10, wherein in step (b) the extract of step (a1) is substantially undiluted.

12. The method of claim 9 or 10, wherein in step (b) the extract of step (a1) is diluted.

13. The method of claim 11 or 12, wherein said extract of step (a1) constitutes 90 to 99% v/v of the entire medium obtained in step (b).

14. The method of any one of claims 11 to 13, wherein said extract of step (a1) constitutes 94 to 98% v/v of the entire medium obtained in step (b).

15. The method of any one of claims 11 to 14, wherein the medium is capable of supporting fungal growth without further dilution steps to reduce the concentration of fungal growth inhibitors.

16. The method of claim 1, the method comprising:
(a2) extracting C5 sugars from lignocellulosic material comprised in BSG via a liquid extraction treatment with water at temperature of between 145° C. and 155° C. and/or for a time up to 70 minutes, preferably for a time up to 50 minutes, preferably at the pressure of 30 to 50 bar, and
(b) combining the so obtained extract with at least one non-carbohydrate nutrient for fungal cultivation,

17. The method of claim 16, wherein the lignocellulosic material comprised in BSG is untreated.

18. The method of claim 17, wherein said material has not been dewatered

19. The method of any one of claims 16 to 18, wherein the (a2) step is performed using water comprising acid, preferably H₂SO₄, at a concentration ranging between 0.4% w/w, to 1.6% w/w.

20. The method of claim 19, wherein the (a2) step is performed using water comprising acid, preferably H₂SO₄, at a concentration ranging between 0.5% w/w to 0.9% w/w.

21. The method of any one of claims 16 to 20, wherein the liquid extraction is performed for a time of 5 to 15 minutes.

22. The method of any one of claims 16 to 21, wherein the liquid extraction is performed at a pH of between 1.0 and 3.0.

23. The method of claim any one of claims 2 to 22, wherein step (a1) or step (a2) is performed at a severity factor of between 2.9 and 3.3.

24. The method of any one of claims 1 to 23, wherein the method further comprises the step (a′) of enzymatic hydrolysis of a solid lignocellulosic residue obtained in step (a1) or in step (a2) with cellulase, separating a liquid product of hydrolysis from a solid residue, and mixing said liquid product with the extract of step (a1) or the extract of step (a2).

25. The method of any one of claims 1 to 24, wherein substantially no sugar beyond that present in the extract of step (a1) or step (a2) or in the liquid product of hydrolysis of step (a′) is added.

26. The method of any one of claims 1 to 25, wherein step (a1) or step (a2) is characterized by the solid load of a reactor of between 4 and 14% w/w.

27. The method of any one of claims 1 to 26, wherein complex C5-polysaccharides constitute at least 50% of all sugars in the extract of step (a1) or step (a2).

28. The method of claim 27, wherein complex C5-polysaccharides constitute at least 65% of all sugars in the extract of step (a1) or step (a2).

29. The method of claim 28, wherein complex C5-polysaccharides constitute at least 80% of all sugars the extract of step (a1) or step (a2).

30. The method of any one of claims 1 to 29, wherein the medium is suitable for cultivation of at least one fungal strain selected from P. pulmonarius and M. rufobrunnea.

31. A fungal fermentation medium, obtainable according to the method of any one of claims 1 to 30.

32. The fungal fermentation medium of claim 31, wherein substantially all the sugar originates from BSG.

33. The fungal fermentation medium of claim 31 or 32, wherein complex C5-polysaccharides constitute at least 50% of all sugars in said medium.

34. The fungal fermentation medium of claim 33, complex C5-polysaccharides constitute at least 65% of all sugars in said medium.

35. The fungal fermentation medium of claim 34, wherein complex C5-polysaccharides constitute at least 80% of all sugars in said medium said medium.

36. Use of the medium of any one of claims 31 to 35 in the fungal culture.

37. A method for producing a fungal biomass, the method comprising the step of submerged fermentation of at least one fungal strain using the fungal fermentation medium of any one of claims claim 31 to 35.

38. The method of claim 37, wherein the at least one fungal strain is an edible fungus.

39. The method of claim 38, wherein the at least one fungal strain is P. pulmonarius P. ostreatus, P. citrinopileatus or P. salmoneostramineus or wherein the at least one fungal strain is M. rufobrunnea, M. esculenta, M. angusticeps or M. deliciosa.

40. A fungal biomass produced according to the method of any one of claims 37 to 39.

41. The fungal biomass of claim 40, characterized by Asp/Asn content of at least 20% of the total amino acid content.

42. Use of the fungal biomass of claim 40 or 41 in production of a food product, preferably fungal-based food product.

43. A food product, preferably a fungal-based food product, prepared using the fungal biomass of claim 40 or 41.
